(19) United States
(12) Patent Application Publication
(10) Pub. No.: US 2026/0207971 A1
(43) Pub. Date: Jul. 23, 2026

(54) Beam Filtration Apparatus and Method for Efficient Scanned Beam Particle Arc Therapy

(71) Applicant: University of Iowa Research Foundation, Iowa CIty, IA (US)

(72) Inventors: Blake Smith, North Liberty, IA (US); Ryan Flynn, Iowa City, IA (US); Daniel Hyer, Iowa City, IA (US)

(21) Appl. No.: 19/140,969

(22) PCT Filed: Dec. 19, 2023
(86) PCT No.: PCT/US2023/084756

§ 371 Date: Jun. 18, 2025

Related U.S. Application Data
(60) Provisional application No. 63/476,030, filed on Dec. 19, 2022.

(51) Int. Cl. A61N 5/10; G21K 1/10

(52) U.S. Cl. CPC A61N 5/1047; G21K 1/10; A61N 2005/1087; A61N 2005/1095

(57) ABSTRACT

A filter for radiation therapy, and other applications, is disclosed. The filter comprises a base and columns of varying heights extending from one face of the base. The column heights are determined by simulating the change in energy distribution of radiation passing through the filter and updating the column heights so as to more accurately match the desired output beam energy distribution. The method of filter design employs an inner loop of faster, but less accurate calculations and an outer loop of slower but more accurate calculations. The column heights for the filter are the last calculated values or the ones which produce the simulated output beam energy distribution which most closely matches the desired output beam energy distribution. The filter can be used in a radiation beam apparatus to treat a patient more quickly.

### BACKGROUND OF THE INVENTION

The present invention relates to devices and methods for filtering radiation, a proton beam as a presented example, to produce the desired energy distribution of radiation for treating a disease.

Pencil beam scanning (PBS) therapy is used to treat diseases, including cancerous tumors, by scanning a beam of high-energy ions throughout a tumor (or target) volume and has been employed as a state-of-the-art technique for proton therapy. Currently, multiple beam energies are required to treat all depths of the target and changing the beam energy during the treatment delivery can take several seconds resulting in several minutes of additional treatment time. Treatment is further complicated when the beam is delivered in a rotational arc, known as rotational arc therapy, implanting the ion beam from multiple directions. While demonstrated for proton therapy to hold superior delivery robustness and improved healthy tissue sparing, the numerous energy changes and low-intensity beams required to cover the target result in prohibitively prolonged treatment times and unstable treatment deliveries.

The combination of active scanning ion therapy while also varying the entrance angle into the patient holds major synergistic potential to drastically reduce normal tissue toxicity, improve target dose conformity (measure of how a volume of a specified dose matches the intended target shape and volume), and mitigate errors associated with static field deliveries. However, the dynamic energy changes and numerous low-intensity beams necessary to deliver an effective rotation arc treatment, such as with PBS proton therapy, result in a clinically prohibitive treatments and prolonged treatment times, which is a particular concern in cases of mobile targets or at a high through-put clinic.

Accordingly, a need arises for techniques that enable robust, and more efficient means to deliver proton arc and expedite changes to the energy of the beam in proton and other ion radiation therapies.

### SUMMARY

Aspects of the disclosure relate to systems and methods for modeling, fabricating, and using a filter which converts the initial energy spectrum of radiation, which can be an ion beam, to a desired spectrum in a manner that is position-dependent within the beam's eye view. The filter comprises a base and a plurality of columns, which are also referred to herein as pillars, protruding (or extending) from one face of the base. One or more of the pillars can be formed from the base (wherein the base can act as a substrate), or one or more of the pillars can sit atop the base. Each column of the plurality of columns has a height and dimensions (e.g., horizontal dimensions such length and width). The dimensions can be perpendicular to the height, or the dimensions can be substantially perpendicular to the height, or the dimensions can be nonperpendicular to the height. The base can be any shape, for example circular, square, octagonal, triangular, or irregular. The columns can also be of any shape, for example, rectangular block, triangular prism, or cylindrical. Each column of the plurality of columns has a location on the base. The dimensions of the columns can all be the same. The heights of the columns can vary as noted by a method of designing the filter. The term “filter” is used herein to refer to one or more example embodiments of a filter in accordance with the present invention, and such a filter can also be referred to as “speleo-filter” due to the similarly in appearance of the filter to that of shaped speleothems in caverns.

The method of designing the filter can start with setting an initial height to each column. In an example, the initial height of each column can be set to a maximum allowable height, which can be a height selected to be the maximum height. Another example would be to set an initial height equal to half of the maximum height. Another example is to initialize each of the column heights to a randomly set value. The heights of the columns can then be varied systematically in a model by a certain step. For example, the step size can be limited to no greater than one third of the maximum allowable column height. In alternative embodiments, the step size limit can decrease as the number of iterations increases. The column heights can be varied using a two-loop process of calculation. The calculation can use an inner loop which employs faster albeit less accurate simulations of an ion beam passing through the filter. The outer loop can employ slower but more accurate methods, such as a Monte Carlo type simulation. The filter's output can be modeled by taking as input (incident onto the base of the filter and passing through the columns) a beam of ions with a known energy distribution, or spectrum. The inner loop can use a coarse approximation to the filter output. This coarse approximation calculation can take the filter shape (base dimensions and column locations, lateral dimensions and heights) and the known energy and spatial distribution of the incident ion beam and calculate a simulated output beam dose distribution. The calculated output beam dose distribution is compared with the desired output beam dose distribution and a new estimate of the column heights is then calculated. This process is iterated until a first selected number of iterations has passed or until the calculated output beam energy and dose distribution matches the desired output beam dose distribution to within a first threshold. After this loop of coarse approximation calculations has been completed, a Monte Carlo simulation of the incident ion beam can be performed including a more accurate model (fine approximation) accounting for all physical properties involved and which provides a more accurate simulated output beam and resulting dose distribution. The first coarse approximation calculations and iterations can then be re-started. The outer/Monte Carlo simulation loops continue, with the inner loops continuing inside the outer loops, until either a second selected number of iterations of the Monte Carlo simulations has been exceed or until the simulated output ion beam dose distribution matches the desired output ion beam dose distribution to within a second threshold. At this point, the filter design process can stop and the heights of the columns are noted. A fabrication of the filter can then occur.

In example embodiments, the filter can be fabricated by three-dimensional printing employing a plastic or organic material such as PMMA and polyacrylate. The filter can be included in an instrument for performing proton beam therapy. The instrument can also comprise a collimation apparatus and the filter can be included either upstream or downstream of the collimation apparatus. The filter can be included in a filter wheel or carousel which can enable rapidly switching filters into the ion beam.

### BRIEF DESCRIPTION OF THE DRAWINGS

So that the manner in which the above and below recited features of example (also referred to as exemplary) embodiments of the present invention can be understood in detail, a more particular description of the example embodiments, briefly summarized above and described below, may be had by reference to example (or exemplary) embodiments, some of which are illustrated in the appended drawings. It is to be noted, however, that the appended drawings illustrate only example embodiments of this invention and the invention may admit to other equally effective embodiments.

FIG. 1 illustrates an example of a filter design.

FIG. 2 illustrates a schematic of the filter optimization process.

FIG. 3 illustrates an example of filter design/optimization

FIG. 4 illustrates an example of filter rendering and optimization

FIG. 5 illustrates an exemplary dose profile curve.

FIG. 6 illustrates a flow diagram for a filter design process.

FIG. 7 illustrates a rendering of an exemplary filter.

FIG. 8 illustrates a rendering of an exemplary filter with ancillary apparatus.

FIG. 9 illustrates a visualization of an exemplary filter in use during an arc therapy treatment.

FIG. 10 illustrates a speleo-filter integrated with an energy-specific collimator known as the dynamic collimation system.

FIG. 11 illustrates a view from below the apparatus and filter shown in FIG. 10.

FIG. 12 illustrates exemplary spread out Bragg (SOBP) peak dose profiles.

FIG. 13 illustrates the distribution of pillar heights for an example speleo-filter used to obtain the 3 cm SOBP dose profile in FIG. 12.

FIG. 14 illustrates the distribution of pillar heights for an example speleo-filter used to obtain the 5 cm SOBP dose profile in FIG. 12.

FIG. 15 illustrates the distribution of pillar heights of pillar heights for an example speleo-filter used to obtain the 7 cm SOBP dose profile in FIG. 12.

FIG. 16 illustrates a simulated dose profile with an exemplary speleo-filter.

FIG. 17 illustrates a simulated dose profile with an exemplary speleo-filter.

FIG. 18 illustrates a simulated dose profile with an exemplary speleo-filter.

FIG. 19 illustrates volume fraction vs relative dose for several exemplary filters.

FIG. 20 illustrates examples of an unshielded filter and a shielded filter that can be made using 3D printing (also referred to as additive manufacturing).

FIG. 21 illustrates the experimental validation of an exemplary speleo-solid filter.

FIG. 22 illustrates the experimental validation of an exemplary speleo-pillar filter.

FIG. 23 illustrates the experimental validation of an exemplary speleo-optimizer filter.

FIG. 24 illustrates a simulated dose profile for an on-axis, full arc exposure for a four-field multi-field intensity modulated proton therapy (IMPT).

FIG. 25 illustrates a simulated dose profile for an on-axis, full arc exposure for exposure with a speleo-filter.

FIG. 26 illustrates a simulated dose volume histogram for the on-axis full arc/four-field IMPT dose plans.

FIG. 27 illustrates a simulated dose profile for an off-axis, half arc exposure for a three-field IMPT.

FIG. 28 illustrates a simulated dose profile for an off-axis, half arc exposure with a speleo-filter.

FIG. 29 illustrates a simulated dose volume histogram for the off-axis half arc/three-field IMPT dose plans.

FIG. 30 illustrates a simulated dose profile for an off-axis, quarter arc exposure for a two-field IMPT.

FIG. 31 illustrates a simulated dose profile for an off-axis, quarter arc exposure with a speleo-filter

FIG. 32 illustrates a simulated dose volume histogram for the off-axis, quarter arc/two-field IMPT dose plans.

FIG. 33 illustrates an exemplary carousel including multiple speleo-filters.

FIG. 34 illustrates a flow chart for the method of designing a filter.

FIG. 35 illustrates a schematic of a computer for executing the method.

FIG. 36 illustrates a method of using the filter for treating a tumor.

Other features of the present example embodiments will be apparent from the Detailed Description that follows.

### DETAILED DESCRIPTION

In the following detailed description of the exemplary embodiments, reference is made to the accompanying drawings, which form a part hereof, and within which are shown by way of illustration specific exemplary embodiments by which the invention may be practiced. It is to be understood that other exemplary embodiments may be utilized and structural changes can be made without departing from the scope of the invention. It should also be understood that a nominal beam energy is clinically described using a mono-energetic nomenclature while the true energy spectrum of an ion beam is polyenergetic, often tightly bound about the nominal beam energy. The methods described in this document adopt such a terminology but do not preclude such an invention whose application or generation is specific to a theoretically true mono-energetic or poly-energetic ion beam. Electrical, mechanical, logical, and structural changes can be made to the example embodiments without departing from the spirit and scope of the present teachings. The following detailed description is therefore not to be taken in a limiting sense, and the scope of the present disclosure is defined by the appended claims and their equivalents.

The present disclosure relates to an ion beam filter which produces a desired multi-energetic ion beam for producing a higher dose in the desired region with a lower dose in healthy tissue.

The device and processes presented in this disclosure can be used to enable the practical implementation of example embodiments of next-generation ion beam therapy technique referred to as pencil beam scanning proton arc therapy. The unique feature and application of exemplary embodiments herein would allow for significantly fewer energy changes across the delivered arc as a patient-specific device optimized to the curvature of the patient and deflection of the scanned beam. The speleo-filter technology would be encompassed by novel treatment planning techniques that can allow for (1) computational optimization of the shape and distribution of the filter to conform uniquely to a patient's anatomy and features of the scanned beam or (2) establishment of a library of generalized (or standardized) filters that could be rapidly interchanged as the arc progresses about the subject.

To the inventors' knowledge, no similar technologies have been proposed to reduce the treatment time of a PBS proton arc delivery by reducing the number of necessary energies with an external device. The only specific works contributed to improving the delivery efficiency of proton arc beams have been algorithmic approaches to prioritize faster energy changes when a treatment is delivered. However, these algorithmic techniques are still limited by the numerous energy changes which are necessary to achieve advantageous dose distributions within a subject. The techniques described in this disclosure circumvent the efficiency concerns of prioritized energy layer switching by fundamentally changing how a proton dose is deposited in a subject from a single beam, resulting in fewer energy changes and thus in a quicker delivery.

Other devices have been proposed to create a spread-out Bragg peak dose distribution from an actively scanned beam line or passive scattering systems. However, these approaches have not been extended to and are incapable of providing the dynamic energy modulation necessary to perform proton arc therapy. The most similar technologies known to the inventors are 3D conformal range modulators which make use of conical rings of material in a pre-ordered array to produce an intended spread-out range in a subject while modifying the distal shape of the intended target through an additional range compensator.

The technologies listed above are multi-part systems that deliver a uniform dose from a single beam direction. As such, these devices have a narrow scope in their application because they only deliver homogenous energy dose profiles from a single direction, preventing their use for arc-style deliveries or non-uniform multi-field treatment techniques. In comparison to the proton arc-enabled speleo-filter design, the spatial distribution of irregularities in the filter can be matched to the range of specified beam angles. In comparison to these other static-field devices, it is not necessary for the speleo-filter design to have a uniform arrangement in the filter to achieve a desired dose distribution in the subject, which can be a specified dose distribution that is determined for the subject. It is also not necessary for the filter to be fully irradiated at any given beam angle to achieve the desired depth modulation and dose distribution.

The optimization approach of the speleo-filter device is unique compared to the other proposed devices listed elsewhere. Currently, 3D conformal modulators are created using a two-step optimization process where (1) the proton fluence from an unobstructed beam is optimized to achieve the desired dose coverage in a subject followed by (2) an optimization of the device to replicate the previously determined fluence. A two-part process was necessary for these other techniques as they lacked sufficient modeling data to accurately account for the differences in the scattering conditions as the filter is planned. The proposed process to optimize the filter shape is done as an integrated process wherein the dose distribution changes in the subject are optimized directly from the changes in the filter. As such, the optimization design inherently accounts for changes in scattering conditions by incorporating advanced radiation transport simulation techniques as part of the dose calculation and optimization.

It is believed that currently no technology or method exists that has enabled PBS proton arc therapy by drastically reducing the number of beam energy changes through actively modifying the beam with an external device. The proposed beam filtration apparatus, called a speleo-filter, is a multi-field, proton arc filter that significantly reduces the number of energy changes required to treat a target by creating a spread-out dose distribution based on the internal anatomy of the intended subject, the range of delivered beam angles, and the shape of the filter. Specifically, the filter is not restricted to a specific beam angle, but rather enables an expedient delivery of PBS treatments across one or more rotational arcs by modifying the delivered beam into a spectrum of different proton energies around the patient. The filter does not rely on supplementary range compensators or other devices to modify the beam energy, is compatible with a non-uniform delivery of protons across a rotational arc and is capable of producing a non-uniform energy distribution of protons to create highly conformal intensity modulated proton beam therapy treatments. The optimization process used to create the filter can be tailored towards generating uniform profiles or creating patient-specific devices for treatment. The filter results in minimal unwanted neutron contamination due to its composition of low-atomic number materials and can be mounted near the patient to achieve superior lateral dose sparing of healthy tissues. The device is mechanically robust and can be mounted on existing PBS collimators to preserve the benefits of energy-specific collimation. Furthermore, while the application presented in this disclosure is specific to proton therapy, the device can be applied to any active scanning or passive scanning ion beam dose delivery system.

Pencil beam scanning (PBS) proton therapy is a form of external beam radiotherapy wherein a narrow beam of protons is raster-scanned across a target, with varying intensities within the scanning pattern, to irradiate the tumor. A single proton beam will traverse a finite distance into the patient, often requiring several different beam energies to adequately cover the extent of the disease. As a result, this treatment technique can provide improved healthy tissue sparing and organ avoidance relative to conventional x-ray therapy for a variety of treatment sites and has been associated with lower toxicities. The next generation of proton therapy entails the delivery of PBS in a continuous arc, referred to as Proton Arc Therapy. It has been shown that the geometric flexibility afforded with an arc delivery combined with the advantages of protons can further improve healthy tissue sparing while increasing the conformity of radiation dose to the intended target. Only recently have the technological developments to these proton systems (including fully rotating gantries and efficient energy layer switching) enabled such a delivery technique.

The speleo-filter invention disclosed herein enables a practical delivery mechanism for proton beam and other ion beam arc therapy treatments by generating multiple beam energies from a single-energy ion source that can be tailored specifically to the subject, herein referred to as the speleo-filter. To date, proton arc therapy remains a practical challenge because the determination and delivery of the necessary energies across the arc often results in a large number of energy changes to adequately cover the target. While some algorithmic approaches exist to prioritize faster energy switching sequencing, the fundamental problem persists and leads to prohibitively long treatment times and technically challenging deliveries. The example embodiments of a speleo-filter in accordance with the invention of the present application relates to an apparatus and method to use one or more beam energy compensators to significantly improve the delivery efficiency by reducing the number of energy changes needed during proton arc therapy.

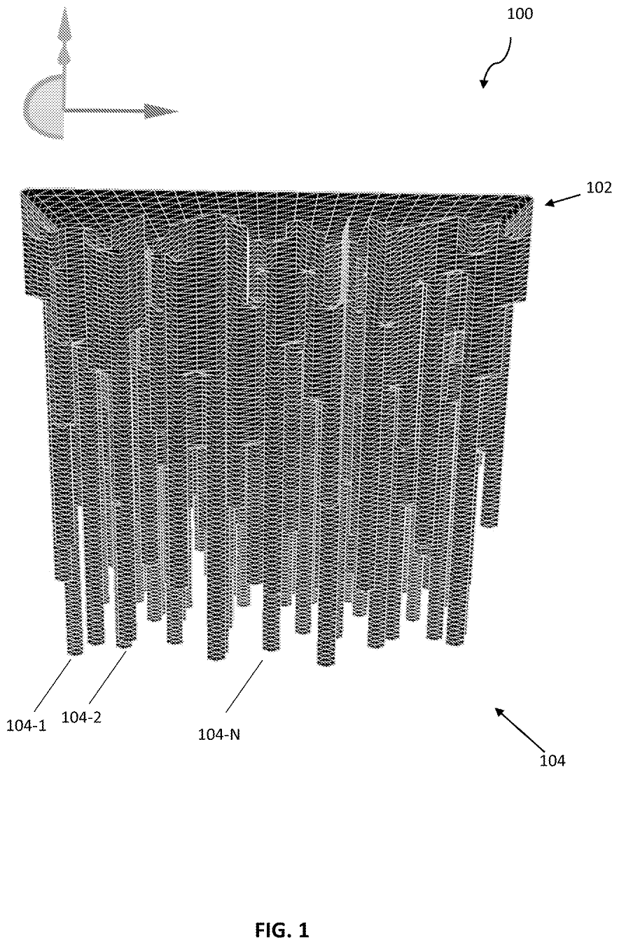

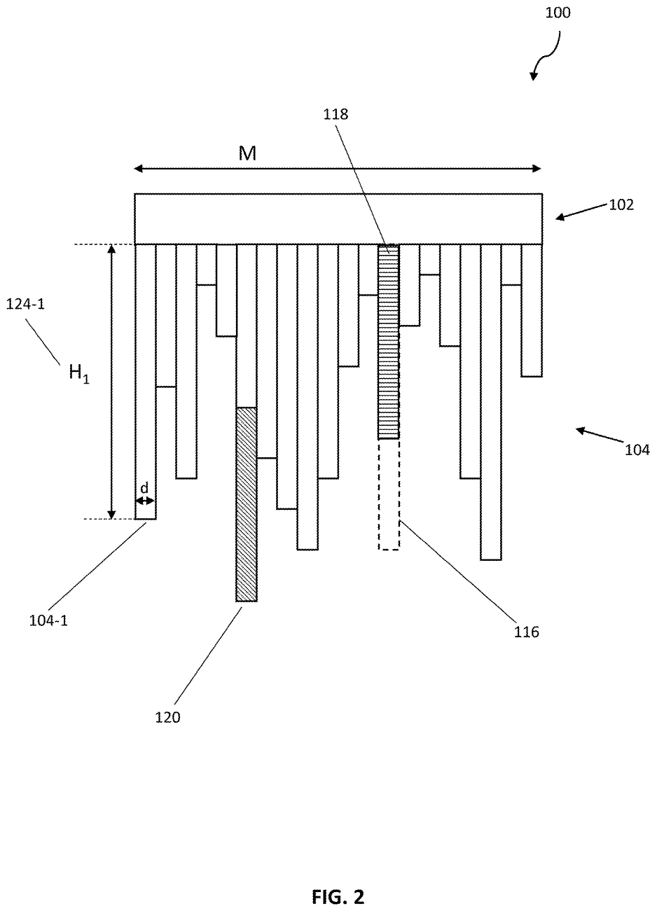

A specialized beam modifier was designed to produce a spatially variant polyenergetic proton spectra that would achieve a desired uniform spread out Bragg peak depth dose profile during PBS proton therapy. Conceptually, the device can be thought of as a heterogenous block of material with pillars of varying height protruding from the base as shown in FIG. 1 and FIG. 2.

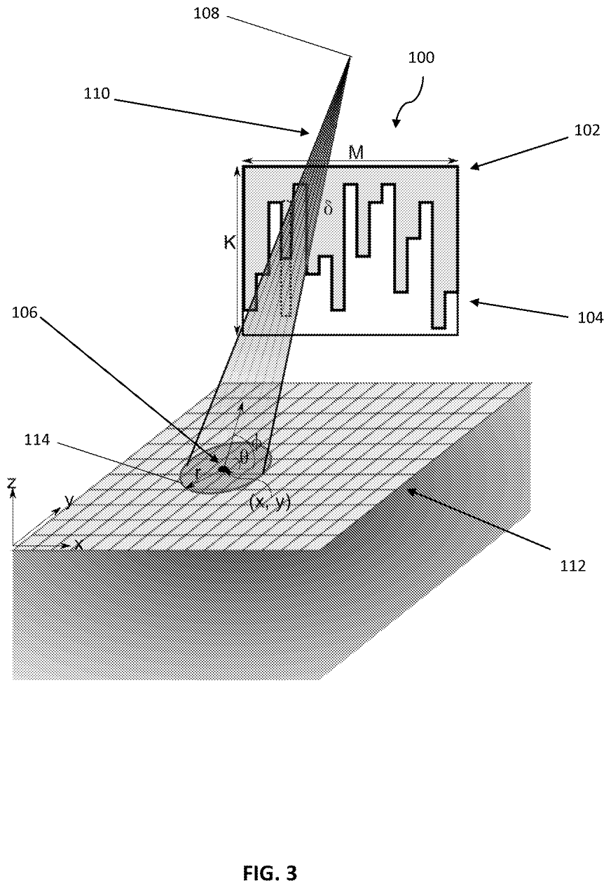

Protons traversing through the filter as shown in FIG. 3 will encounter differing amounts of the filter material due to the varying of the height of each of the pillars. The amount of material that each proton encounters depends on the particular trajectory through the filter. In some cases, a proton can entirely traverse the space occupied by the filter in the empty space between adjacent pillars whereas other trajectories result in a proton continuously interacting within the filter material. When scanned with a proton beam, a targeted depth dose profile can then be produced using a proper arrangement of the pillars that adheres to a specific distribution of projected path lengths throughout the filter as illustrated in FIG. 3.

The distribution of proton energies resulting from the different path lengths throughout the filter can occur in a relatively small area so that each beamlet traversing across the filter undergoes sufficient spatially variant degradation. The repetition of this pattern can be spatially invariant when superimposed across an entire field. As presented in this example, the distribution of columns and their heights were optimized using a hybrid direct parameter and simulated annealing approach where the height of each column was iteratively adjusted in series across the entire filter defined using an M×N×K binary matrix, F, where each column of the filter was discretized among the set of elements, S={1 . . . K}. The column height at each index m and n was optimized by,

$${F}^{i + 1} ( m , n , k ) = \{ \begin{matrix}{F}_{( m , n , k )}^{i} + 1 ∀ k ∈ \{ S \vert {k}_{r} + 1 \leq k \leq {k}_{r} + 1 + Δ \} & if Δ > 0 and {\text{?}}_{obj} ( {F}^{i + 1} ) < {\text{?}}_{obj} ( {F}^{i} ) \\ {F}_{( m , n , k )}^{i} + 1 ∀ k ∈ \{ S \vert {k}_{r} + Δ \leq k \leq {k}_{r} \} & if Δ < 0 and {\text{?}}_{obj} ( {F}^{i + 1} ) < {\text{?}}_{obj} ( {F}^{i} ) \\ {F}_{( m , n , k )}^{i} ∀ k ∈ \{ S \} & {\text{?}}_{obj} ( {F}^{i} ) < {\text{?}}_{obj} ( {F}^{i + 1} )\end{matrix} \qquad ( 1 )$$

$$where ,$$

$${k}_{r} = max ( K \vert {F}_{m , n , k}^{i} = 1 ) , \qquad ( 2 )$$

The search size, Δ, is determined from the iteration number, i, of the optimization starting, in this example, at the equivalent length to one-third of the maximum filter height and continuously reducing to the size of a single discretized filter element in the matrix, F,

$$Δ = \frac{K}{( i + 1 )} . \qquad ( 3 )$$

Filters can be optimized to produce a spatially distributed areal proton energy spectrum across the entire intended treatment field that would produce a specified spread out Bragg peak (SOBP) goal with width, ∈, and depth, ξ, using a modified RECT function, Π, defined for each iteration of the optimization, I, and a user-defined penalty function, β, which can be partitioned into multiple piecewise components, from 1 to T parts, to help shape the distal and proximal penumbra of the SOBP distribution from a distance, η from the edge of the desired field width by applying a distance-specific penalty weighting factor, b. A dose goal and penalty function can be defined as,

$${\prod }_{i} ( \frac{z - ξ}{ϵ} ) = \{ \begin{matrix}1 & if ξ - ϵ / 2 \leq z \leq ξ + ϵ / 2 , \\ 0 & otherwise\end{matrix} \qquad ( 4 )$$

$$and ,$$

$$β ( \frac{z - ξ}{ϵ} ) = \{ \begin{matrix}1 & if z < ξ - ϵ / 2 and {PDD}_{w} ( z ) > 1 \\ 1 & if ξ - ϵ / 2 \leq z \leq ξ + ϵ / 2 \\ {b}_{1} & if z > ξ + ϵ / 2 + {η}_{1} \\ & ⋮ \\ {b}_{T} & if z > ξ + ϵ / 2 + {η}_{T} \\ 0 & otherwise\end{matrix} . \qquad ( 5 )$$

The depth dose distribution resulting from the filter filled with numerous heterogeneities is difficult to precisely model analytically as a large contribution to the percent depth dose (PDD)

$${PDD}_{w} ( x , y , z ) = \frac{{\int }_{0}^{2 π} {\int }_{0}^{{r}_{i}} {PDD}_{0} ( z + δ ( x + r \cdot cos ( θ ) \cdot y + r \cdot sin ( θ ) ) ) dr d θ}{π {r}_{i}^{2}} , \qquad ( 6 )$$

shape is based on the path length differences and scattering conditions between adjacent paths throughout the filter. However, to a first-order approximation, it was assumed for this example that the PDD could be described by the distribution of proton energies about a radius of the PDD's central axis, where the areal-averaged percent depth dose, PDD_{w}, along the central axis intersecting at (x, y) in the plane of the radiation isocenter is the average of the neighboring PDD curves based on the regional filter heterogeneities projected to a circle of radius, rᵢ, from the effective source point during the i^{th} iteration of the filter optimization. For computational efficiency, the resulting PDD profiles along a ray can be back projected through a water-equivalent amount, δ, of the filter, F,

$$δ ( {x}_{0} , {y}_{0} ) = {\int }_{τ = 0}^{T} ρ ( {x}_{0} + τ \cdot cos ( θ ) sin ( φ ) , {y}_{0} + τ \cdot sin ( θ ) sin ( φ ) , {z}_{0} + τ \cdot cos ( φ ) ) d τ , \qquad ( 7 )$$

as illustrated in FIG. 3. The corresponding PDDs can be determined by scaling the depth axis of a reference PDD curve, PDD₀, by the amount of water-equivalent range traversed through the filter. The reference PDD curve can be generated at the start of the optimization for an unobstructed proton beam incident on a water phantom using the targeted proton beam energy. For this example, each iteration of the optimization computes an objective function, [symbol] , by computing the squared differences between the weighted PDD at a point to the targeted PDD profile,

$${\text{?}}_{obj} ( {F}^{i} ) = {\int }_{x = {x}_{0}}^{X} {\int }_{y = {y}_{0}}^{Y} {\int }_{0}^{Z} {( ( {PDD}_{w} ( x , y , z ) - {\prod }_{i} ( \frac{z - ξ}{ϵ} ) ) \cdot β ( z ) )}^{2} dx dy dz . \qquad ( 8 )$$

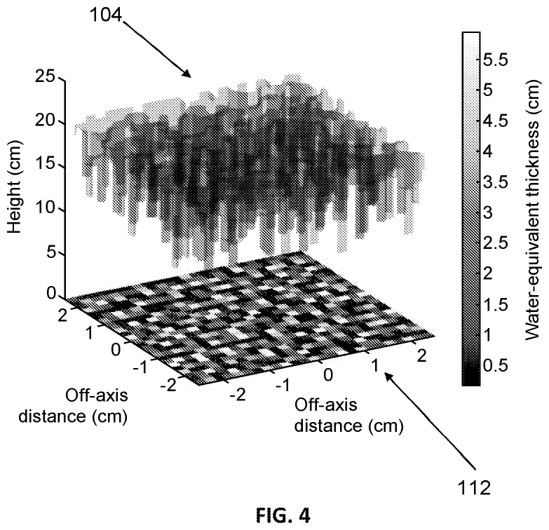

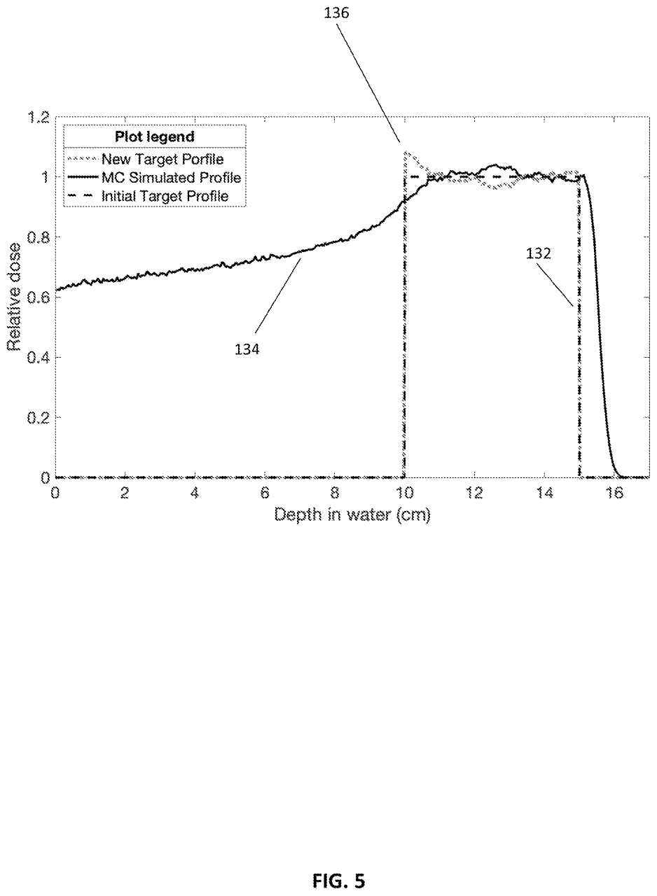

The above equations and formalism have been implemented and run to generate a design for 5 cm×5 cm filters with target SOBP widths of 3 cm, 4 cm, 5 cm and 7 cm in R2019a version of MATLAB. In an example, a filter could be designed using rectangular pillars in which each pillar had a basal dimension of 1 mm by 1 mm, leading to a filter of 50×50=2500 pillars. The determination of the resultant PDD due to the distribution water equivalent thickness (WET) projected through the filter, while an acceptable first-order approximation, is insufficient to precisely model the changing scattering conditions throughout the filter. Adequately modeling these changes is difficult, if not impractical, using beamlet-based methods. Monte Carlo simulated transport is an appropriate approach to accurately model the dosimetric changes due to physical geometric changes in the filter. However, it may be impractical to perform a Monte Carlo calculation to assess each discrete filter perturbation. Instead, Monte Carlo calculations may be performed in stages throughout the optimization process once it appears the current filter design has converged upon a stable objective function value (such as every 10-30 stages as demonstrated in this work). An in-water percent depth dose (PDD) profile can be calculated by transporting the intended field of mono-energetic protons through the filter at the current design stage and can then be used to generate a new objective function goal from the current goal, thereby inherently accounting for the intra-filter scatter modeling discrepancies as the design evolves. The initial objective function goal can be modeled as a RECT function spanning the desired SOBP and is subsequently updated by,

$${\prod }_{i + 1} ( z ) = \frac{{\prod }_{i} ( z )}{{PDD}_{MC , i} ( z )} . \qquad ( 9 )$$

where PDD_{MC,i} is the Monte Carlo-generated PDD profile from the filter at the current stage of design at each depth, z, in water. The mathematical projection of an exemplary filter's water equivalent thickness (WET) at the radiation isocenter plane and the corresponding PDD profile along the central axis is shown in FIG. 4 and FIG. 5, respectively. These results can then be used to generate a new depth-dose objective function subsequently used in the optimization of the filter design.

Monte Carlo simulations can be performed using the Monte Carlo toolkit Geant4 (Version 4.1005) or other suitable software. In the example using Geant4, simulation executables were compiled from native or customized class structures in C++ that were called by a filter optimization program from MATLAB® and were run in parallel with that program. Optimizations and Monte Carlo simulations were performed locally on a quad-core 2.8 GHZ Intel® Core™ i7 processor. For this work, the Monte Carlo framework served multiple purposes including:
1) providing updated objective functions to the optimizer,
2) evaluating the uniformity of the delivered SOBP dose distributions from the optimized filters,
3) benchmarking the computational toolkit against measured profiles, and
4) studying rotational delivery to demonstrate the use of this technology in proton arc therapy.

The charged particle energy loss for all simulations was determined using the standard g4 h-phy QGSP_BIC_HP hadronic and g4em-standard opt4 electromagnetic transport physics package with the physics transport constraints listed in Table I. Nuclear interaction and uncharged radiation transport physics were registered to the run manager throughout the G4VModularPhysicsList class using the standard QGSP_BIC_HP package available in Geant4. Absorbed dose to water was tallied in a voxel array using the G4Primitive Scorer to sum energy depositions and record the statistical variance.

In an example, a custom divergent point source was modeled after the Ion Beam Applications (IBA) Dedicated Nozzle (DN) Proteus® One beam line at the Miami Cancer Center (MCI) in Gainesville, Florida. Other proton sources can also be used for filter design calculations. Spot divergence can be modeled following Monte Carlo techniques by back-projecting commissioning fluence distributions along the central axis and calculating an angular fluence pattern at the effective source position. Beam line-specific parameters for the IBA DN system and modeling of the beam deflection during scanning using an X- and Y-bending magnet can be adopted from an experimentally benchmarked Dynamic Collimation Monte Carlo (DCMC) simulation package. This package accurately models the double-focused alignment of the trimmers in the dynamic collimation system (DCS) to each scanning magnet. Using these models, the beamline optics can be mathematically accounted for in the source definition, resulting in excellent spot profile and PDD agreement on axis and off axis. The use of this modeling framework also enables a compatible framework with the dynamic collimation system (DCS), a state-of-the-art PBS collimator.

TABLE I
Electromagnetic option 4 transport variables and values used for the simulation
of proton and electron transport during DCS GRID simulations in Geant4

| | EM | | |
|---|---|---|---|
| Parameters | Option 4 | | Comments |
| MinEnergy | 100 | eV | Minimum kinetic energy of tables |
| NumberOfBinsPerDecade | 20 | | number of bins per decade of a table |
| dRoverRange | 0.2 | | electron allowed step length relative to current |
| | | | range |
| finalRange | 10 | μm | electron article tracking cut (energy threshold) |
| | | | limit |
| dRoverRangeMuHad | 0.1 | | hadron allowed step length relative to current |
| | | | range |
| finalRangeMuHad | 20 | μm | hadron article tracking cut limit (energy |
| | | | threshold) |

| | | |
|---|---|---|
| McsRangeFactor | 0.2 | electron MCS step limit |

The speleo-filters can be modeled as an array of, for example, 2 mm×2 mm×0.05 mm filter elements contained within the 3D matrix, F. An output file can be generated following the optimization which can compartmentalize the volume of the matrix as dry air at standard temperature and pressure and the filter material can be modeled as acrylic, though other materials can also be used. The extent of the matrix, not necessarily the tip of the outmost filter element, was offset from a 40 cm×40 cm×40 cm water phantom with the beam line isocenter placed at the water tank surface. Each simulation utilized a scanned field of mono-energetic protons across the entire surface of the filter with a 1 mm lateral spot spacing unless otherwise noted. High-energy, peripheral spray from protons exiting the lateral extent of the filter were absorbed by modeling a simple brass aperture, during optimization. Alternatively, the device could be positioned within an external collimation device where low-energy scatter is absorbed in the collimating components.

A 150 MeV proton beam, for example, can be used to optimize and evaluate the quality of the optimized filter design. In a demonstration, a generalization of the filter to produce the desired spread out Bragg peak (SOBP) was evaluated using 110 MeV, 130 MeV and 170 MeV. The filter was nominally centered along the beam's central axis and was displaced laterally in 1 mm increments while evaluating the sensitivity to alignment errors. Single- and multi-dimensional tallies were used to quantify the dose distributions delivered using these filters. The absorbed dose-to-water tallies used to compute PDD profiles were computed in a cylindrical stack 1 cm radius with a depth resolution of 0.5 mm. Single field, SOBP uniformity was evaluated using dose grid resolution of 1.5 mm.

### Arc-Based Treatment Planning Study

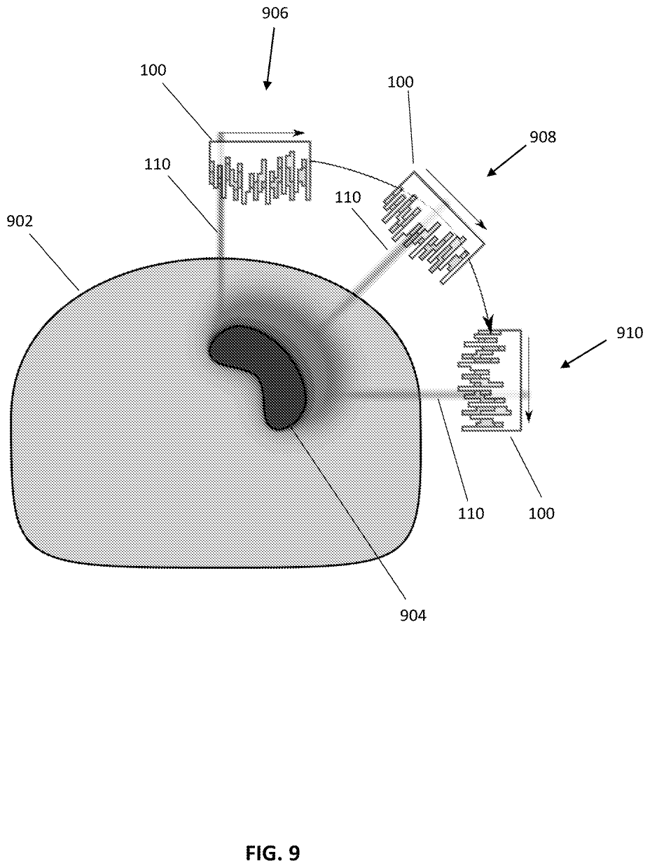

An exemplary full and a partial pencil beam scan (PBS) proton arc therapy treatment delivery were simulated using an exemplary 5 cm speleo-filter as shown schematically in FIG. 9. Exemplary treatment plans were generated to cover an example 5.8 cm diameter target that was embedded within a 25 cm diameter cylindrical water phantom along the central axis, for which a full arc was used, or offset laterally by 5 cm utilizing only a partial, 180° arc. Each treatment was optimized from a set of beamlets delivered in a 5 cm square field with a 2.5 mm spot spacing delivered across the entire arc, which was approximated in 1° increments. A 150 MeV proton beam was used in combination with the 5 cm speleo-filter to treat the central target while three plans were created for the off-axis target that utilized the three beam energies listed in Table II. A set of corresponding multi-field intensity modulated proton therapy (IMPT) treatment plans were also created for comparison that used an equivalent scanning field size, lateral spot spacing, and 2.5 MeV energy spacing.

TABLE II
Energy layer and beam angle distribution for the exemplary on-
and off-axis treatment plans. Multi-field IMPT (intensity modulated
proton therapy) treatments were performed with four beam angles
for on-axis deliveries and with three and two beam angles for
off-axis deliveries. A full 360° arc with a nominal 150
MeV proton beam was used for the on-axis case whereas the off-
axis partial arc treatment was organized into three arc angles
across a 90° angle range. The corresponding half arc for
this case mirrored the beamlets across the horizontal axis
Off-axis, 3-energy, 5 cm Speleo-Filter

| | arc 1 | arc 2 | arc 3 |
|---|---|---|---|
| Beam angles (deg) | 0-53 | 53-74 | 74-90 |
| Target extent (cm) | 10-11.3 | 11.3-12.7 | 12.7-14 |
| Ē (MeV) | 119.1 | 128.9 | 136.9 |

| | |
|---|---|
| Field 1 | Field 2 |

On-axis, IMPT delivery

| | | |
|---|---|---|
| Beam angles (deg) | 0 | 90 |
| Target extent (cm) | 10-15 | 10-15 |
| Ē (MeV) | 100-150 (21) | 100-150 (21) |

Off-axis IMTP delivery

| | | |
|---|---|---|
| Beam angles (deg) | 0 | 90 |
| Target extent (cm) | 9-14 | 5-10 |
| Ē (MeV) | 109-141.5 (14) | 77-117 (17) |

Individual beamlets were ran with 1×10⁵ histories using the Geant4 simulation executable to generate a library of beamlet profiles with a 1 mm dose grid resolution whose intensities were optimized using a least-squares minimization approach for IMPT,

$$F = {\sum }_{k = 1}^{K} ( \frac{1}{{T}_{k}} {\sum }_{i ∈ {τ}_{k}} [ {β}_{k}^{+} {c}_{( 0 , ∞ )}^{2} ( {d}_{i} - {d}_{i , k}^{+} ) + {β}_{k}^{-} {c}_{( - ∞ , 0 )}^{2} ( {d}_{i} - {d}_{i , k}^{-} ) + {β}_{k}^{{V}_{k}^{+}} {c}_{( 0 , Δ {D}_{k}^{{V}_{k}^{+}} )}^{2} ( {d}_{i} - {d}_{i , k}^{+} ) + {β}_{k}^{{V}_{k}^{-}} {c}_{( 0 , Δ {D}_{k}^{{V}_{k}^{-}} )}^{2} ( {d}_{i} - {d}_{i , k}^{-} ) ] ) , \qquad ( 10 )$$

$$and ,$$

$${c}_{( a , b )} ( x ) = \{ \begin{matrix}x & if a \leq x \leq b \\ 0 & else\end{matrix} . \qquad ( 11 )$$

where the specific variables are summarized in Table III. Four regions were considered for the purposes of this optimization exercise. As an example, the 5 cm diameter target was optimized so that 95% of its volume received a dose of 50 Gy. The skin was defined as the inner 5 mm region from the outer perimeter. High-dose conformity was quantified from a 10 mm rind of healthy tissue immediately adjacent to the intended target, and the remaining material was categorized as general body.

TABLE III
Definitions of variables used in Equation 8

| Variable | Description |
|---|---|
| τₖ, Tₖ | Subset voxel indices and number of voxels, respectively, |
| | that are inside tissue k with k ≡ ξ denoting the target |
| ${d}_{i , k}^{\pm } , {β}_{k}^{\pm }$ | Dose threshold for voxel i and penalty weight, respec- tively, for overdose(+) and underdose(−) penalties for |
| | tissue k |
| ${d}_{i , k}^{V \pm } , {V}_{k}^{\pm } , {β}_{k}^{{V}_{k}^{\pm }}$ | Dose threshold and penalty weight, respectively, for dose- volume overdose(+) and underdose(−)penalties for tissue |
| | k |
| $Δ {D}_{k}^{{V}_{k}^{\pm }}$ | Difference between the dose planned and desired to Vₖ^{±} in the cumulative dose-volume histogram for tissue k |

### Conceptual Prototype Production

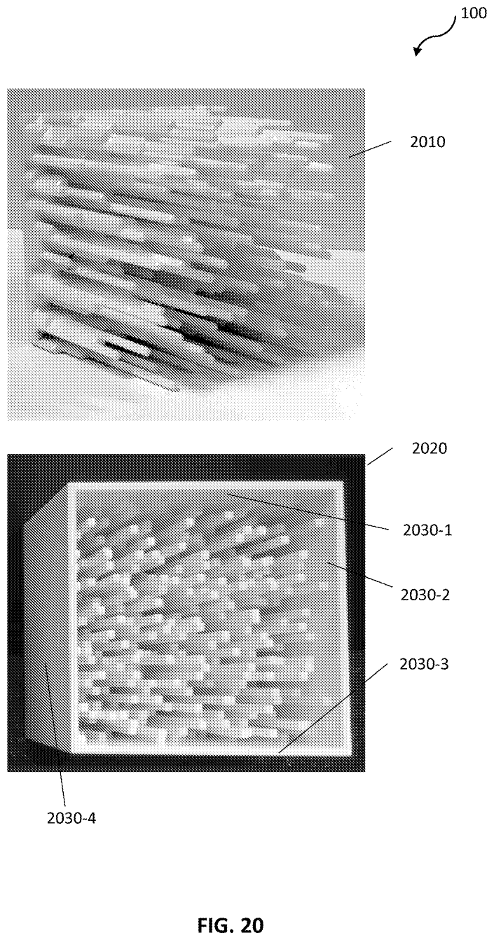

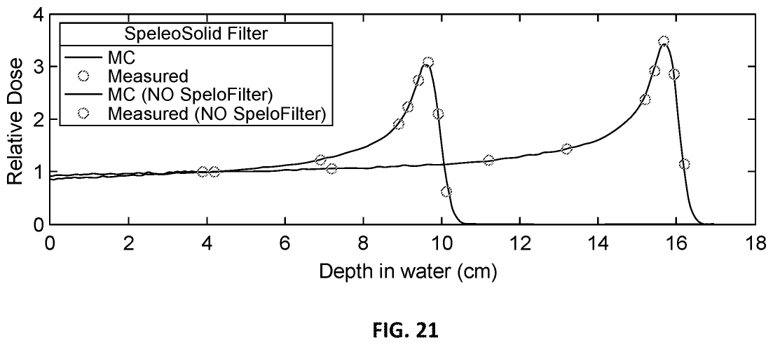

An exemplary filter was fabricated with an exemplary optimization plan to produce a 5 cm SOBP. The filter was 3D printed by Stratasys™ printing using the PolyJet™ printing technique, although other printers, fabrication techniques, or materials can also be used. The exemplary filters were printed from a MATLAB®-generated.stl file from the triangulation of the optimized surface. A glossy print that omitted the use of supportive wax can be used. Alternatively, a conventional print mode can also be employed. Other 3D printing options can also be employed, as is known to those skilled in the art. Given the numerous thin, delicate features, care must be taken to evaluate fabrication options. The exemplary filters described here were printed using a white VeroClear™ material, which is a photopolymer that mimics the properties of polymethyl methacrylate (PMMA) and has a nominal density of 1.19 g/cm³. Other example materials which can also be used to fabricate a speleo-filter include VeroClear™, VeroUltraClear™, VeroUltraClearS™, VeroUltra™, polylactic acid (PLA), Somos® 9120, Somos® WaterShed, Somos® WaterClear Ultra, Somo® BioClear, nylon, carbon fiber, ABS-M30i, ULTEM™ resin or other polytherimide thermoplastics, photopolymers, acrylic, brass, tungsten, nickel, beryllium, copper, aluminum, SolidWater®, Virtual Water™, lead, graphite, and paraffin wax. The manufacturing accuracy of these exemplary filters can be benchmarked from a high-resolution CT wherein this study utilized a 0.6 mm slice thickness. These scans were acquired on a Siemens Biograph™ PET-CT scanner. A treatment planning system can be used to generate the outer surface from the scanned filter. As an example, the outer surface of the example filters was generated by thresholding the surface of the filter and contouring each scan in Pinnacle® (Version 16.2), which can than be exported as a DICOM structure set and compared against the digital design of the filter. This was demonstrated using a custom MATLAB® application that overlay the CT-drawn surface contour against the original optimized filter design and quantify the manufacturing accuracy by computing the DICE similarity coefficient between the two surface contours and reporting the mean Hausdorff distance, although other metrics can be used. FIG. 20 shows a set of exemplary filters (unshielded and shielded) in accordance with example embodiments of the present invention, with graphs depicting their experimental validation (FIGS. 21-23).

### Optimized SOBP Uniformity

A Monte Carlo analysis can be performed to obtain a further SOBP optimization for a filter which can include a higher number of simulation histories to minimize simulation error. Each exemplary speleo-filter was benchmarked against the desired SOBP width, center, and uniformity. Other metrics can also be used the characterize and benchmark the dosimetric characteristics of a filter. Table IV lists the results from the Monte Carlo simulations of all four example filters in addition to the scenarios where a positional error was incorporated to evaluate the dosimetric sensitivity of the optimized filter to positional offsets.

TABLE VI
Spread-out Bragg peak characteristics for the 3 cm, 5 cm and
7 cm speleo-filters used during a uniform PBS delivery including
the center of the SOBP, the width, and uniformity defined by the
maximum deviation and the 95^{th} percentile uniformity. Intentional
offsets are listed filter offsets are upstream towards the
ion source, unless otherwise noted.

| | | | Spread-out Bragg Peak | | |
|---|---|---|---|---|---|
| | | | | Max. | 95% |
| | Offset | Center | Width | Deviation | Uniformity |
| Filter | (cm) | (cm) | (cm) | (%) | (%) |
| 3 cm SOBP | 5 | 13.35 | 3.15 | 2.45 | 1.96 |
| 5 cm SOBP | 5 | 12.41 | 5.1 | 2.53 | 1.74 |
| | 10 | 12.41 | 5.25 | 2.87 | 2.15 |
| | 20 | 12.37 | 5.24 | 3.40 | 2.41 |
| | 1 mm lat | 12.42 | 5.21 | 2.56 | 2.02 |
| 7 cm SOBP | 5 | 11.44 | 7.42 | 3.56 | 2.49 |

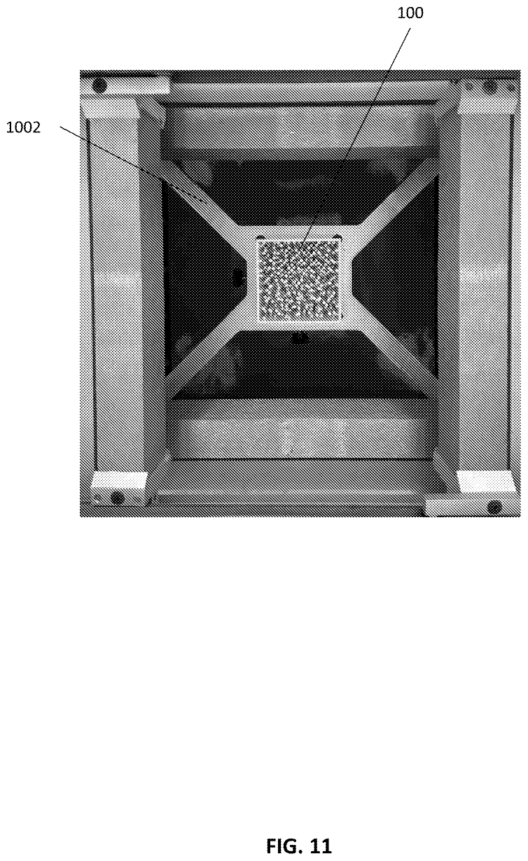

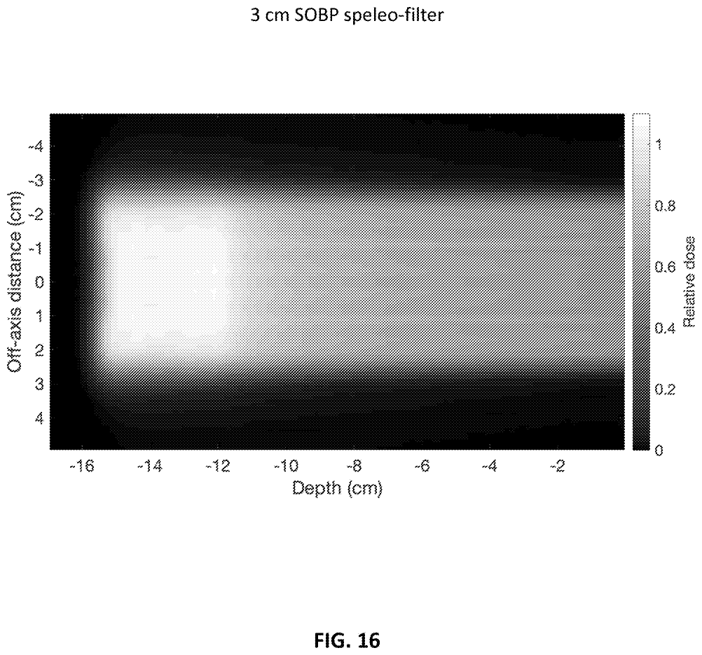

Volumetric simulations of the SOBP volumes can be calculated to demonstrate the three-dimensional dosimetric characteristics as demonstrated for the 3 cm, 5 cm and 7 cm speleo-filters as well as an offset 5 cm speleo-filter positioned 10 cm above the isocenter. As shown for the dose distributions presented in FIGS. 11-19, dose distributions can be from uniformly scanned fields. Two-dimensional depth dose profiles and the dose-volume histograms are presented in FIGS. 16-19. For a relative comparison, the simulated volumes can be normalized to the mean dose within the targeted SOBP based on the nominal range and the inner 80% lateral field width as done for this example. The D_{95%} coverage, D_{2%}, and uniformity are listed in Table V. For this study, each filter achieved an SOBP uniformity to within 10%. Increasing the filter SOBP width can reduce the SOBP dose uniformity; 95% of the voxels within the 7 cm SOBP target in this example were within 7.55% compared to 6.21% from the smaller 3 cm speleo-filter. All the exemplary filters achieved over 90% coverage for the SOBP target.

TABLE V
Dose-volume histogram metrics for the simulated 3D dose-
volumes resulting from a 150 MeV uniformly scanned proton
beam across 3 cm, 5 cm and 7 cm speleo-filters

| Filter | Offset (cm) | D_{95%} | D_{2%} (%) | 95% Uniformity |
|---|---|---|---|---|
| 3 cm SOBP | 5 | 0.937 | 104 | 6.21% |
| 5 cm SOBP | 5 | 0.932 | 107 | 7.39% |
| 5 cm SOBP | 10 | 0.914 | 109 | 9.40% |
| 7 cm SOBP | 5 | 0.931 | 107 | 7.55% |

### Experimental Quality Control

A quality control step can be incorporated to benchmark the structural accuracy of the 3D printing against the intended design following its optimization. A high-resolution CT scan can be acquired following the 3D printing of the filter, shown in FIG. 20. Great agreement was observed between the weight-windowed auto contour of the filter's surface against the computerized model in this example. A density-weighted Sørensen-Dice similarity coefficient can be calculated to compare the volume overlap of a fabricated filter with its originally intended design. This comparison can be achieved by weighting each pixel by the CT-determined density for only voxels containing at least 25% of the expected density of the VeroClear™ material. The density-weighted Sørensen-Dice coefficient was found to be 0.978 between the CT scan and the computerized model for this exemplary filter. Similarly, the Hausdorff distance was only evaluated for pixels that had a CT-measured density of at least 25% of the expected VeroClear™ material. The maximum observed 95%, 98% and 99% Hausdorff distances in this example were 0.50 mm, 0.71 mm and 1.00 mm, respectively. For this study it was found that the CT-determined density was within 0.84% of the manufacturer-stated density.

Experimental Validation of Technology within a Clinical Treatment Unit

The dosimetric properties of the speleo-filters have been experimentally verified within a clinical proton beamline at the Miami Cancer Institute's (MCI) IBA (Ion Beam Applications, Louvain-la-neuve, Belgium) Proteus Plus Dedicated Nozzle (DN) PBS system. A set of three example embodiments of speleo-filters in accordance with the present invention were created and mounted within an example custom holder that was secured to the DCS in place of the external range shifter, upstream of the collimating components shown in FIG. 20 of exemplary embodiments of spleo-filters. Each filter served a specific purpose to (1) benchmark the radiological properties (e.g., density, etc., that affect the effective path length, arrangement, and shape) using a solid block (speleo-filter referred to as SpeleoSolid), benchmark Monte Carlo transport methods and experimental sensitivity (SpeleoPillar), and experimentally demonstrate, via proof-of-principle, the planning and integration of an optimized speleo-filter (SpeleoOptimized). As the SpeleoSolid filter comprised of a uniform, solid block, differences in material density would have dominated any discrepancies between the Monte Carlo simulated profile and what was measured while being insensitive to small alignment offsets. The SpeleoPillar filter was designed with a repeating pattern of 50 mm and 3 mm pillars to further refine material definition as well as study the alignment sensitivity of the speleo-filters to the incident proton beam. These features were used in the analysis of the profiles to discriminate between setup errors and nominal material composition differences between the simulated and measurement conditions. The spatial characteristics of the SpeleoOptimized filter were optimized to produce a 5 cm uniform depth dose distribution using the mathematical methods discussed in this application.

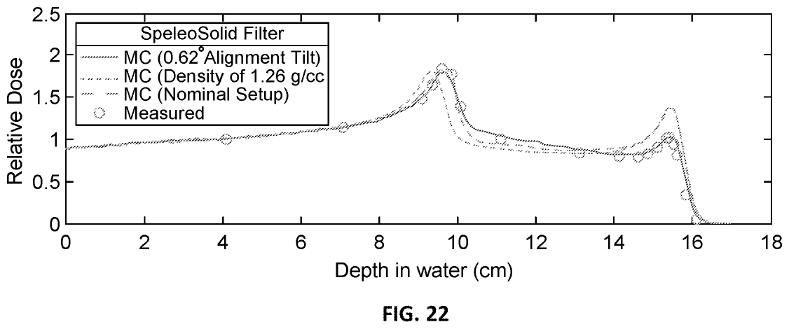

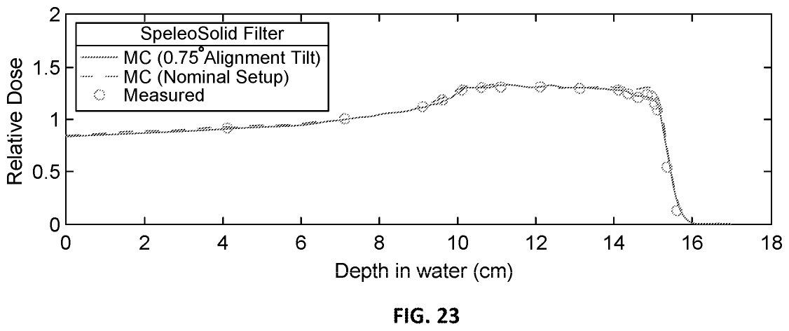

Dose measurements have been acquired using the IBA DN system at the Miami Cancer Institute along the central axis at specified depths within a water phantom for each prototype speleo-filter. FIGS. 21-23 below show the measured and simulated dose profiles used to validate the Monte Carlo model for the SpeleoSolid, SpeleoPillar, and the SpeleoOptimized speleo-filters. The initial pristine profile that was measured for an unfiltered 150 MeV beam shows excellent agreement with the simulated profile. All measurement points were found to match the Monte Carlo simulated SpeleoSolid profile to within a 1%/1 mm gamma criteria. Additional Monte Carlo simulations were ran by changing the nominal density and orientation of the SpeleoSolid filter to investigate the impact on the resultant depth dose profile. As shown in FIG. 22, changes to the material density caused the relative separation of the peaks to change while any deflection of the speleo-filter normal to the direction of the scanned beam caused changes to the relative intensity of the two peaks. A nominal density of 1.19 g/cm³ with a speleo-filter setup error of 0.62 degrees rotated about the X- and Y-plane resulted in a Monte-Carlo generated profile that matched all measurement points to within a 2%/1 mm gamma criteria. The measured and Monte Carlo simulated profiles for the SpeleoOptimized speleo-filter are plotted in FIG. 23 assuming the nominal material density and 0.75 degree angled off set. All measurement points matched with the Monte Carlo simulated profile to within a 1%/1 mm gamma criteria and achieved a uniformity of 3% of the mean across the dose profile.

### Treatment Planning Comparison

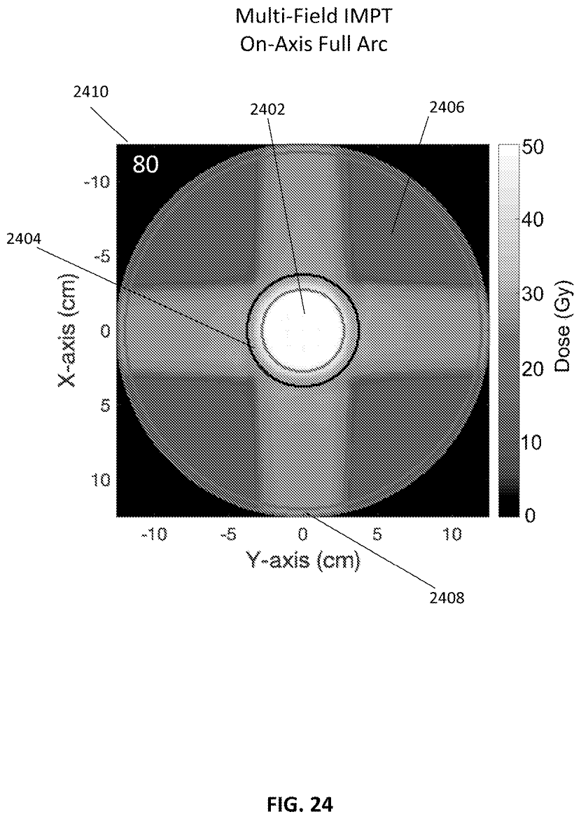

Treatment plans can be generated and compared for standard multi-field IMPT and rotational arc treatments using, for example, a 5 cm SOBP speleo-filter. Dose profiles for each plan are shown in FIGS. 24-32. IMPT and speleo-filter treatment plans can be directly compared against one another based on their planning geometries and beam arrangement similarities. In this example, off-axis target treatments were planned to use either the three-field (180 degree span angle) or two-field (90 degree span angle) IMPT technique that was matched against a half-arc treatment or a quarter-arc treatment using the speleo-filter. A Four-field IMPT and full-arc Speleo treatment plan were paired against one another for a centrally located target. Exemplary plans optimized using a speleo-filter resulted in comparable or superior treatment planning quality as summarized in Table VI and FIGS. 24-32. Depending on the field arrangement and arc span, the high dose conformity can remain similar among IMPT and arc treatments delivered using a speleo-filter ranging, in this example, between 1.059-1.088 and 1.036-1.06, respectively, with smaller span angles and fewer arcs resulting in a poorer high-dose conformity. Low-dose conformity was improved by 17%, 15% and 4% for the quarter-, half-, and full-arc deliveries, respectively, using a rotational arc and a speleo-filter. Similarly, the maximum skin dose and integral body dose can all be improved using the rotational arc delivery and speleo-filter over a multi-field IMPT techniques. The maximum skin dose sparing ranged between 81%, 48% and 7% for the full-, half-, and partial-arcs while the integral dose sparing was improved by 25%, 23% and 17% among the four-, three-, and two-field IMPT treatments, respectively.

TABLE VI
Dose profile summary of each delivery after treating a target to 50 Gy. Full and four-
field IMPT treatments were used to treat a centrally located target while the partial
arcs, two- and three-field IMPT plans were used to treat an off-axis target.

| Delivery | Number of | Conformity Index | | Skin Max | Integral Dose |
|---|---|---|---|---|---|
| Method | Energy Changes | 90% | 50% | (Gy) | (10⁻³ Gym³) |
| 4-field | 80 | 1.059 | 1.617 | 15.23 | 1.800 |
| IMPT | | | | | |
| 3-field | 42 | 1.070 | 1.930 | 24.27 | 1.255 |
| IMPT | | | | | |
| 2-field | 26 | 1.088 | 2.487 | 26.85 | 1.121 |
| IMPT | | | | | |
| Full arc | 0 | 1.068 | 1.555 | 2.78 | 1.342 |
| Half arc | 4 | 1.036 | 1.643 | 12.62 | 0.972 |
| Quarter arc | 2 | 1.082 | 2.063 | 24.86 | 0.929 |

The largest improvement observed in these examples between the arc-treatments using the speleo-filter and standard multi-field IMPT technique was the reduction in the number of energy layers (or energy changes) necessary to deliver the plan. Both on- and off-axis treatments were delivered using a fraction of the number of energy changes necessary for contemporary multi-field IMPT. As a result of the cylindrical symmetry when the target was centrally located, a single nominal energy was sufficient when delivered with the speleo-filter. Off-axis, superior treatment plans could be produced using an order of magnitude fewer beam energies than were necessary for the IMPT technique.

### DISCUSSION

In thinking of speleothems, a cave structure formed by the deposition of minerals from water, speleo-filters produce a milliscopic, spatially variant energy fluence that can be optimized to produce a desired Spread-out Bragg peak (SOBP) dose distribution, which was demonstrated for this work to produce an exemplary uniform dose profile. The dose distribution can be customized by adjusting the relative heights of the individual spokes or columns for a variety of applications through which this technology is intended to benefit. A notable application is a carousel that can rapidly transition the filters throughout the treatment. These filters can also be customized for patient-specific applications allowing for precise multi-field IMPT and rotational arc deliveries. It is also practical to see this technology enabling FLASH particle therapy, such as in PBS as only a single energy is necessary to deliver a composite 3D dose distribution at a minimal cost to the incident proton fluence. The down-stream design of the filters is compatible with external collimation, preserving a powerful upcoming feature in modern PBS. Additionally, the mechanical simplicity of this technology enables a seamless integration with most modern PBS beam lines that is not vendor specific nor reliant on secondary technologies. The example described herein is presented using an IBA DN system and a DCS as an example. While this technology has been presented here with a direct application to proton therapy, the technology can also be applicable to other ion beams treatment modalities including helium and carbon-ion.

Combining pencil beam scanning with an arc-style delivery has been demonstrated to hold large synergistic potential, creating both more conformal and robust treatment plans. However, the distribution and sequencing of energy layers has been a difficult feat to solve as only a single energy can be delivered at a time. Even with sophisticated energy switching algorithms, the total delivery time can be significant (several hundred seconds to more than one thousand seconds) due to the numerous energy changes required to cover the target across a wide angular span. This invention of a speleo-filter offers an alternative paradigm to PBS arc therapy that can shorten the treatment time while maintaining a similar, and potentially improved, degree of plan quality compared to traditional IMPT approaches.

The speleo-filter effectively augments a mono-energetic scanned proton beam to transform it into a polyenergetic proton beamlet while preserving the inherent directivity of the proton beamlet. This is in contrast to other methods of creating Spread-Out Bragg Peaks using a modulator wheel or ridge filter. These alternative methods require placement upstream in the beam path which tends to prohibit the direct application for active scanning techniques given that a spectrum of proton energies will result in a different distribution of momentum changes as the beam subsequently travels through a set of bending magnets. (The ion optics will cause additional distortion in the energy distribution of the proton beam if the proton beam has a distribution of energies as the beam is steered.) External ridge filters have been proposed to deliver an SOBP for some radiotherapy applications, such as FLASH, using a series of repeated conical or pyramidal structures. However, such designs suffer in that their resulting dose distributions have repetitious hot and cold patterns with a relatively low dose homogeneity over the central SOBP region. While this can be suitable for FLASH deliveries so long as a minimum dose rate is achieved, the same cannot be said for complex IMPT or PBS arc deliveries that depend on a uniform target dose and minimization of extraneous hot spots from occurring within the surrounding healthy tissues. The speleo-filter can incorporate an element of randomness into its design in order to prevent repetitious patterns of hot and cold spots occurring within the high-dose region. The spokes protruding from the base responsible for the SOBP profile are spatially distinct across the entire filter.

Clearly, reducing the number of energy layers necessary to deliver a treatment will reduce the total treatment time and enable a higher throughout volume. This gain is especially true for PBS arc therapy, which can require more energy changes than a conventional multi-field SFUD or IMPT treatment. The incorporation of the speleo-filter dramatically cuts down on the necessary energy changes. Instead of delivering a series of individual beamlet energies, a sequence of filters, each optimized to produce a particular dose profile, are employed as the gantry rotates around the patient. Multiple filters can be mounted within a carousel that can be interchanged within a fraction of a second. An alternative technique has been proposed to actively produce an SOBP using a variable ridge filter by offsetting the alignment of a set of mirrored ridges. An unfortunate consequence of the mirrored ridge design results in a bulky device with limited achievable SOBP widths and poor flatness while still relying on multiple SOBP profiles to create large, composite SOBPs. The speleo-filter design is unique in that these filters are not limited to an achievable SOBP width. It is predicted that so long as a fine enough 3D printing resolution is available, larger uniform SOBPs should be possible. This limit stems from the necessary spatial distribution of proton energies produced by the speleo-filter within a finite, but discrete, areal region. Lower SOBP widths require less areal resolution than larger SOBPs.

Conventional IMPT methods also prove unreliable for proton arc, often resulting in underivable treatment plans. As the beam is scanned, often redundantly for many locations, a series of bending magnets produce an energy-specific, time-variant magnetic field to cover the lateral extent of the intended target at each depth. These spot positions and energy layers are preselected in the planning process where the intensity of the beam at each location is optimized to achieve the desired dose goals. An issue of utilizing conventional IMPT delivery methods to deliver proton arc is the minimum assignable intensity or dose contribution from a particular beam spot. This limit represents the lowest deliverable output from the system; as the number of beam spots increases for treatments using continuous arcs about the patient, the average output per beam spot decreases. This has prompted recent works to incorporate spot sparsity, or the portion of beam spots with no dose contribution, as an optimization parameter often resulting in poorer plan quality consisting of a non-uniform treatment volume and unacceptable high-dose streaking into nearby healthy tissues or organs. Conventional planning methods for proton arc therapy have been shown to generate undeliverable treatments that require beam output intensities below minimum thresholds to ensure delivery robustness.

In contrast to conventional PBS where the superposition of multiple beamlets of varying energy and intensity produce the desired depth dose profile, a single speleo-filter will produce a polyenergetic proton beamlet that is comprised of the necessary relative abundance of proton energies to achieve the equivalent depth dose profile produced by conventional methods. This allows the freedom in the optimization process to deliver a virtually unrestricted spatially variable depth dose profile delivered using as few as one beam energy. The down-stream placement of the filter from the bending magnets combined with the utility of a mono-energetic beam preserves the spot scanning and high-precision intensity modulation capabilities afforded by PBS [36]. Without the need to superimpose multiple proton beamlets of different energy, each restricted to a minimum MU delivery, a beamlet delivered from a speleo-filter can achieve the same relative depth dose profile without the same limitations

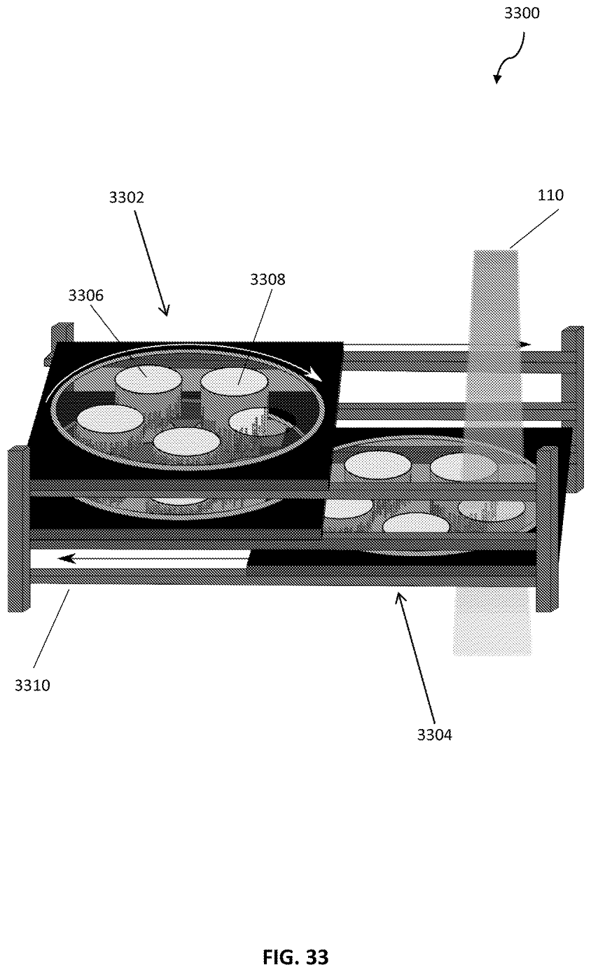

Speleo-filters can be generalized or customized for a particular application. Unlike fixed ridge filters, the shape and resulting proton distribution can be optimized to cover nearly any dosimetric goal. This opens the door towards patient-specific devices that can be manufactured to tailor a treatment uniquely to the geometry of the patient. The optimization and 3D-printing process can be generalized to accommodate such personalized treatment requests. Alternatively, a set of standard filters can be produced and set within a high-speed carousel, such as depicted in FIG. 33, which can enable switching of filters within a fraction of a second. Such a set of general filters can easily be fabricated for specific treatments that are modeled in a treatment planning system that provides speleo-filter-specific profile optimization. In addition to their generalizability among a multitude of treatment options, the 3D-printing process can also use materials with a very low neutron contamination in contrast to high-Z materials often found in modulator wheels and ridge filters which can pose radiation safety risks to staff and patients over time. Finally, the compact design of these filters enables their integration with state-of-the-art PBS collimators. As demonstrated in this work, a custom mounting prototype can secure the filter components just upstream the collimating blades to preserve the lateral penumbral shaping capabilities of these collimators, which has significant potential to enhance target conformity and healthy tissue sparing when combined with an arc-style delivery.

FIG. 1 depicts a computer rendering of a filter 100, also referred to as speleo-filter 100, during the design. In this example, the filter 100 has a base 102, from which extend a group of columns 104. The base can be flat as presented in this example or curved. This group 104 is composed of many individual columns 104-1, 104-2, etc, which are different heights. The group 104 covers the entire length and width of the base 102. In this example, the base 102 is square, but other shapes are also possible, depending on the beam shape and the desired end dose required. In this example, the columns 104-1, 104-2, etc. are also square, but, again, other shapes for the columns can also be used to achieve similar results. The columns 104-1, 104-2, etc. can be defined by a location on the base plane, by a height, and by a dimension or dimensions parallel to the plane of the base 102. Thus, square columns can be defined by their location on the base, a height, and a width of the column. Rectangular columns can be defined by a location, a height, and also by a width and a length. Analogously, circular columns can be defined by a location, a height, and a diameter or a radius and other shapes can be defined as well, like hexagonal, triangular, etc. The columns may or may not extend in the same direction.

FIG. 2 illustrates a sideview profile of a cut through a filter. The filter 100 being modeled has a base 102 and many columns 104. The columns can have material added 120 or subtracted 116 to leave a resulting column material 118. For modelling, each column (e.g. first column 104-1) can have an associated height (e.g. first height H₁ 124-1). The addition or subtraction of material takes place in each iteration of the optimization calculations. The physical fabrication of the filter can take place only after the iterations converge upon the desired solution. The minimum column width can be modeled by a dimension d which could be a column radius or diameter for round columns, or a column width for square columns. For simplicity, the column dimension perpendicular to the height is shown as uniform for every column. In reality, it is possible to assign to each column a particular dimension which need not be the same for every column, but can vary across the filter. In this figure, only a single planar cut-through section of the filter 100 is depicted for simplicity. In this example, the maximum height of the first column 104-1 can be denoted as H₁ 124-1. and the width of the base 102 is M. The smallest width of a column is d.

FIG. 3 also depicts a sideview cut-through profile of one layer of the filter as well as illustrating how a depth dose profile can be calculated. The filter 100 comprises a base 102 and a set of columns/pillars 104. In this illustration of a model during the optimization phase of filter design, the filter material can be removed from a column 116 (dashed line, behind ion beam 110) or added to a column (not shown in this figure, but in FIG. 2) during each iteration of the filter optimization process. The simulated material can be added or subtracted to an individual column based on calculations to achieve the desired depth profile of the implanted ions. In an example, a desired profile can be a spread-out Bragg peak (SOBP) of protons in human tissue. The greatest possible height of a column 104 is shown in the figure as K, but each individual column 104-1, 104-2, etc, will have its own height H₁, H₂, etc. and that height will depend on the location of the center of the column on the base. In principle, the horizontal dimension(s) of each column/pillar can also vary; however, for easy of fabrication of demonstration units, the pillar widths have been kept constant for all pillars. In the examples shown in this application, the base 102 is square and thus defined by a single length, M.

The depth dose profiles at each point (x, y) 106 in the evaluation plane 112 are determined from the product of all the resulting depth profiles within a circular region 114, which are ray-traced through the filter 100 to determine the effective amount of range pullback applied at each point in the evaluation plane 112. The origin 108 of the ion/proton beam 110 is shown in this example as a point source and the proton beam 110 is thus depicted as a diverging beam, although the beam geometry is not so constrained in actual practice. The proton beam 110 travels through the filter 100 at an oblique angle. The proton beam 110 can be diverging, converging, or collimated and this figure is not intended to limit how the beam passes through the filter. In other examples, the proton beam 110 can pass through the filter perpendicularly to the base 102 and the beam 110 can be collimated before or after traversing through the filter.

FIG. 4 illustrates more details of the optimization scheme used to create an exemplary speleo-filter. The columns 104 are modelled as having different heights 124 which vary across the width and length of the filter 100. For clarity, the base 102 of the filter is omitted in this figure. At a target plane 112, the dose of a beam of protons 110 is modelled. The grid shows the off-axis distance in two horizontal directions (X and Y) and the height is for the height of the columns. The water-equivalent thickness bar shows thicker sections in lighter grey and thinner sections in darker grey. The target plane 112 grid shows, in this example, for each 5 mm by 5 mm square the water-equivalent thickness (WET). The scale bar starts at 0 cm, and runs to 5.5 cm WET. The filters are created from a set of pillars 104, shown in semi-transparent shading, whose heights 124 are optimized to produce a specified spread out Bragg peak (SOBP) uniformly across the entire treatment field 112 at a height of 0 cm. The resultant percent depth doses (PDDs) are calculated based on the project water-equivalent thickness through the filter at each pixel.

FIG. 5 illustrates an exemplary dose vs depth (in water) curve calculated for one full iteration of the optimization method. The graph plots the relative absorbed dose as a function of depth in water as a proxy for human tissue. Throughout the optimization, the depth dose goal can frequently change to reflect the changing scattering conditions within the filter. This example curve shows an initial penalty profile 132, a Monte Carlo (MC) simulated profile 134 given the existing filter, and the new target percent depth dose profile 136. The new target profile can be calculated from the current target profile, the filter parameters (dimensions and materials parameters), and the MC simulated profile. Each iteration of this process can take the new target profile as the next penalty profile, followed by a MC simulation, and the next iteration of the profile being calculated.

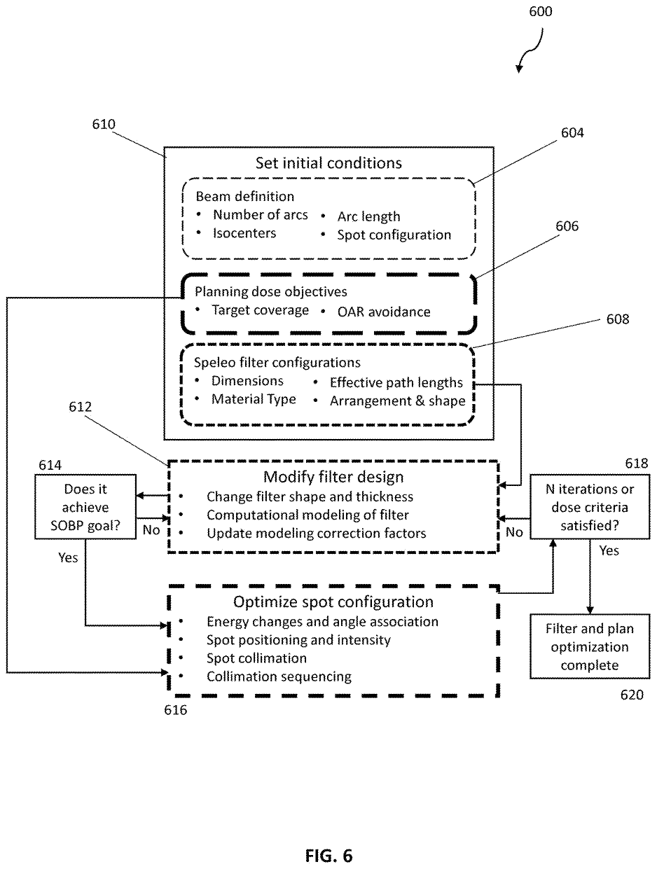

FIG. 6 illustrates a method to optimize a filter for a treatment plan. A unique feature of this method includes that the filter can be designed directly within the planning process while advanced computational methods continuously improve the calculation model due to changing geometric conditions. The method of filter design 600 starts with a set of initial conditions 610. These initial conditions comprise beam definition 604, planning dose objectives 606, and filter configurations 608. The beam definition 604 can comprise the number of arcs, the isocenters, the arc length, the angle of entry, the spot configuration, the beam scanning delivery pattern, and the type of ion(s) being used, amongst other aspects. The planning dose objectives 606 can comprise the target coverage and the organs at risk (OAR) avoidance, as well as other objectives such as healthy tissue sparing. The speleo-filter configurations 608 can comprise the filter dimensions (columns and base, including length, width, depth of base, and the column height and dimension(s) perpendicular to the column height at each location on the base), material type, effective path lengths, and the arrangement and shape of these materials. The speleo-filter configurations 608 can also be set by the user or by the program based on the planning beam definition 604 or the planning dose objectives 606. These initial conditions are fed into the second step 612, and some are also fed into the later step optimize spot configuration 616. At step 610, the initial conditions are set. At step 612, the filter design is modified. The filter designed shape and design dimensions can be changed. The filter design can be computationally modelled and the model can be updated by including correction factors. After the filter design has been modified and the model updated, an evaluation step 614 takes place. At the evaluation step 614, the results of the model are compared to the goal of the target dose (e.g. SOBP). If the goal has been achieved, then the method moves to the next step of spot configuration optimization 616. If the modelled dose does not achieve the goal, then the filter design is again modified. The filter design modification step 612 is iterated until the evaluation step 614 meets the goal or until a present number of iterations is completed. The optimization spot configuration step 616 can use some of the information (e.g. planning dose objectives 606) from the initial conditions step 610. At the optimize spot configuration step 616, the modelled proton/ion beam is perturbed to change, for instance, the energy, the angle association, the spot positioning, the spot intensity, the spot collimation, or the collimation sequencing or any other parameter required. At the following evaluation step 618, either the process has undergone a set number of iterations or the dose criteria have been satisfied including the spot optimization modifications. If neither of these conditions are met, then the method starts again at modify filter design 612, using the most recent set of conditions (e.g., beam definition, planning dose objectives, achieved dose distribution, and speleo-filter configurations). Eventually either the number of iterations has been exceeded or the dose criteria have been satisfied. Once this occurs, the filter design plan optimization is completed 620.

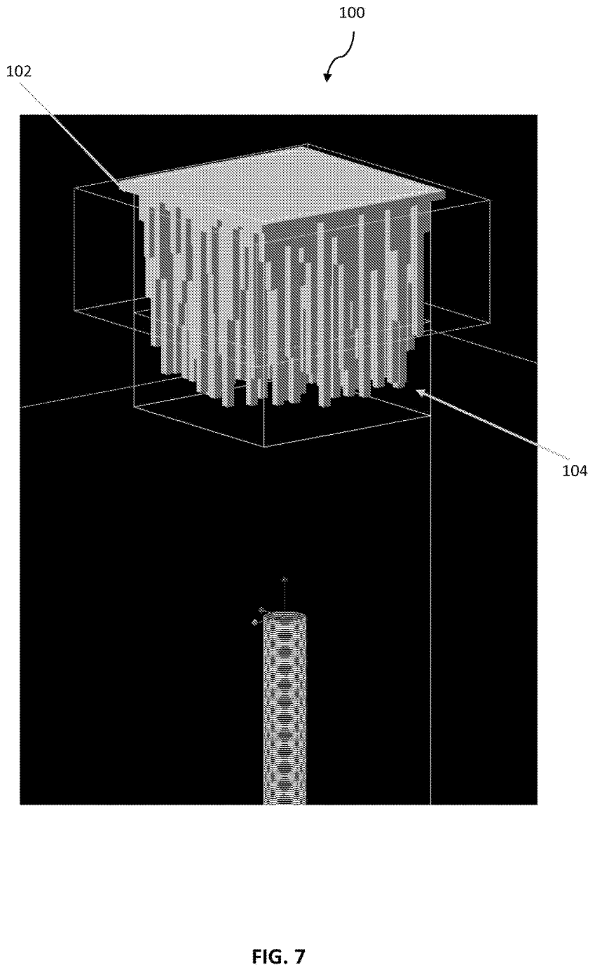

FIG. 7 illustrates a rendering of a designed filter 100. The filter 100 has the base 102 and columns/pillars 104 attached to the base 102. The columns 104 have heights 124 which can vary in different locations across the base 102. In this example, the Geant4 package was used to produce a visual rendering of a 5 cm speleo-filter and surrounding aperture set 5 cm above the surface of a water phantom where a PDD tally is calculated from a 5 cm×5 cm irradiated field.

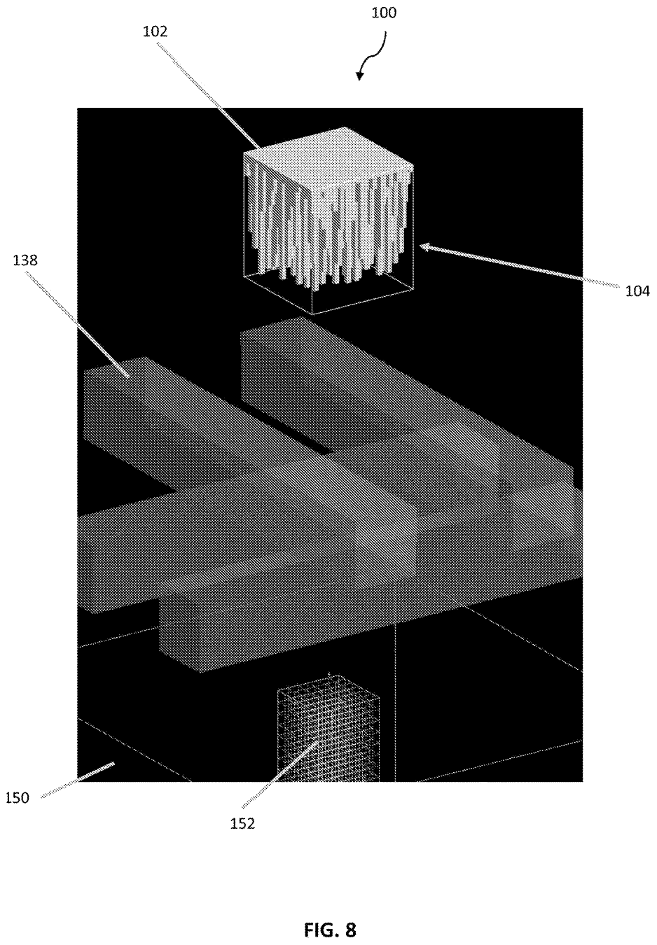

FIG. 8 illustrates a Geant4 visual rendering for a Monte Carlo model of the DCS collimating trimmers 138 with the speleo-filter inserted at the height of an accessory slot in the DCS. A phantom 150 with a three-dimensional dose tally 152 was used to determine the resulting dose profile generated by the filter 100 and the incident beam.

FIG. 9 schematically illustrates a proton arc treatment delivered using pencil beam scanning and a speleo-filter 100. A single scanned proton beam 110 is scanned across the filter 100 at a first location 906 (and with a first direction of entry). The pencil beam enters the human tissue 902, and is absorbed at the target location 904. The filter 100 and beam 110 can then be moved to a second location 908 and the patient 902 can be irradiated a second time from the second direction and location 908. Then the filter 100 and beam 110 can be moved to third location 910, and the patient can be irradiated a third time from the third direction and location 910. The patient 902 can be irradiated by the pencil proton beam 110 at many location and from many angles or during a continuous rotational arc. The overall goal is to create an absorbed dose at the target 904 such that the tumor (or other target) will absorb the bulk of the protons there, which will inhibit function or kill the tumor. At each of the locations, the beam 110 is scanned across the filter 100, and the entire beam plus filter apparatus is moved to irradiate the patient from different directions, typically along an arc. In this example, the patient 902 is irradiated at three locations and from three different angles of entry 906, 908, and 910. Of course, different beam entry angles at the same location or the same beam entry angle at different locations are options which can also be chosen when treating a patient. Additionally, the beam can be fixed while the filter is fixed or moved while the patient is rotated. The idea behind PBS is to treat the intended target in the subject from multiple beam directions. The placement of a properly designed speleo-filter 100 into the beam 110 reduces the number of energy changes necessary during an arc by modifying the energy distribution of the incident proton beam 110 prior to entry into the patient 902. The speleo-filter 100 can be optimized to the shape of the intended target 904 from one or more projections across different beam angles within the arc.

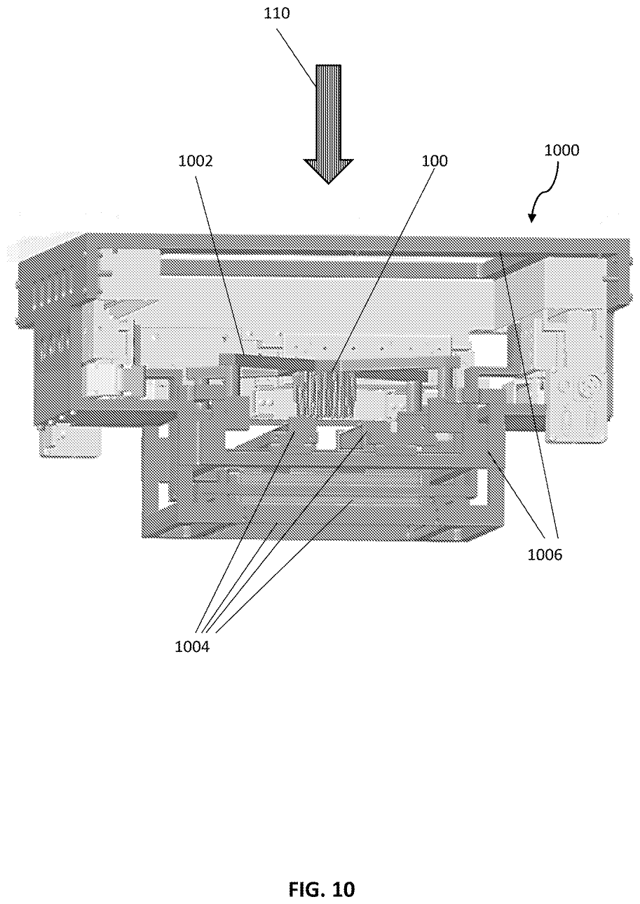

FIG. 10 illustrates an example dynamic collimation system (DCS) 1000 with a speleo-filter 100 in the center. In example embodiments, the filter 100 can be unshielded, wherein a shielded filter and unshielded filter are differentiated below with respect to FIG. 20. CAD model rendering shows an accessory mount for an external range shifter replaced with a custom speleo-filter holder 1002 upstream of the set of collimating trimmers 1004. The proton beam 110 is incident upon the speleofilter 100 located in a holder 1002. The collimators 1004 collimate the beam by blocking and absorbing non-collimated protons. A frame 1006 holds the entire apparatus together.

FIG. 11 shows a view of the DCS of FIG. 10 retaining a speleo-filter 100 in place, wherein the filer 100 is shielded.

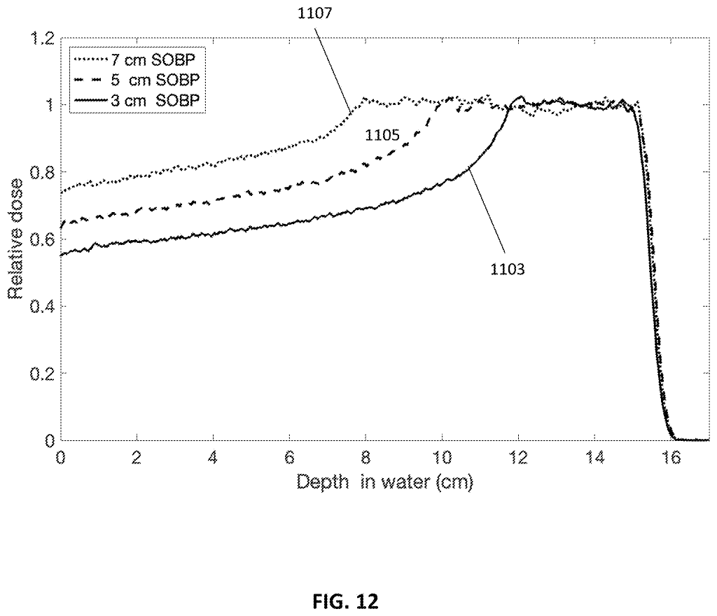

FIG. 12 illustrates the relative dose as a function of depth in water for three different speleo-filters. The graph depicts the spread-out Bragg peak profiles for a nominal 3 cm filter 1103, a 5 cm filter 1105, and a 7 cm filter 1107. These profiles are along the central axis and were simulated using the Geant4 for a uniform field delivery using a pencil beam scan.

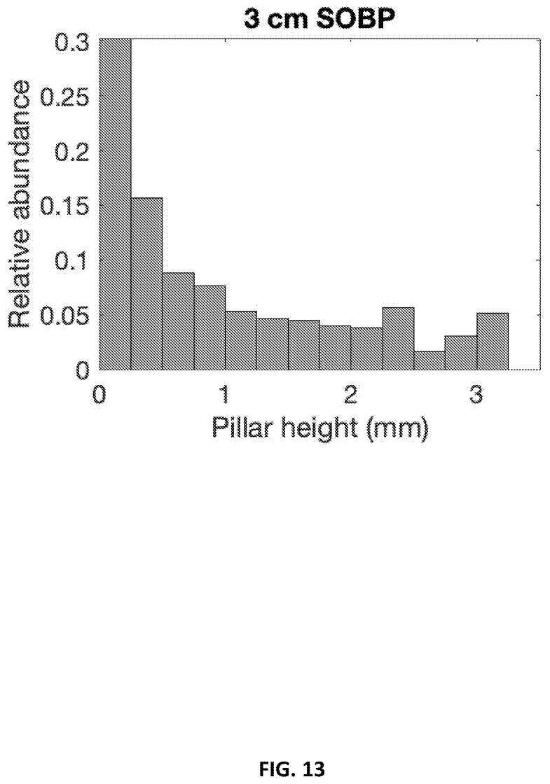

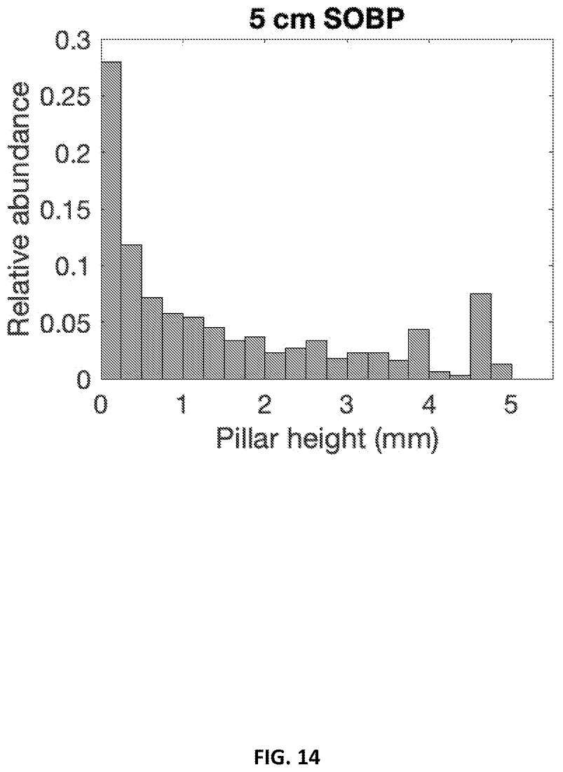

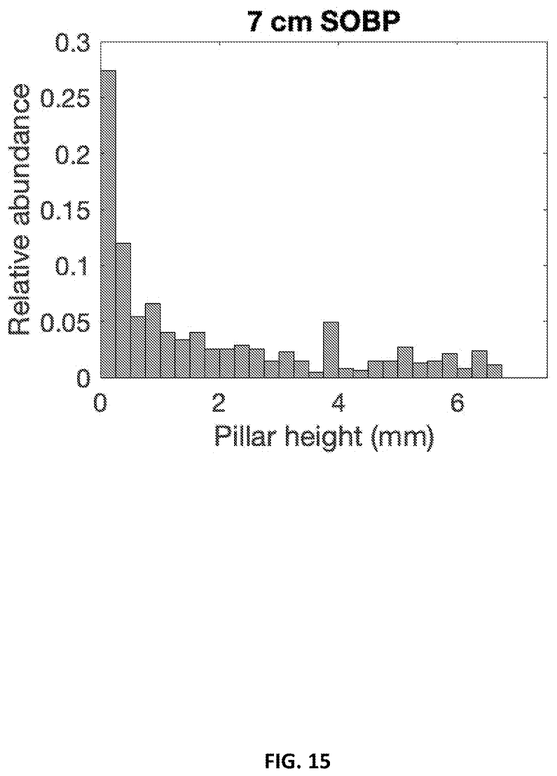

FIG. 13, FIG. 14, and FIG. 15 depict graphs representative of the distribution of pillar heights for each speleo-filter used to obtain the dose profiles shown in the graph of FIG. 12. The pillar heights have corresponding relative abundance of proton energies to achieve the equivalent depth dose profile.

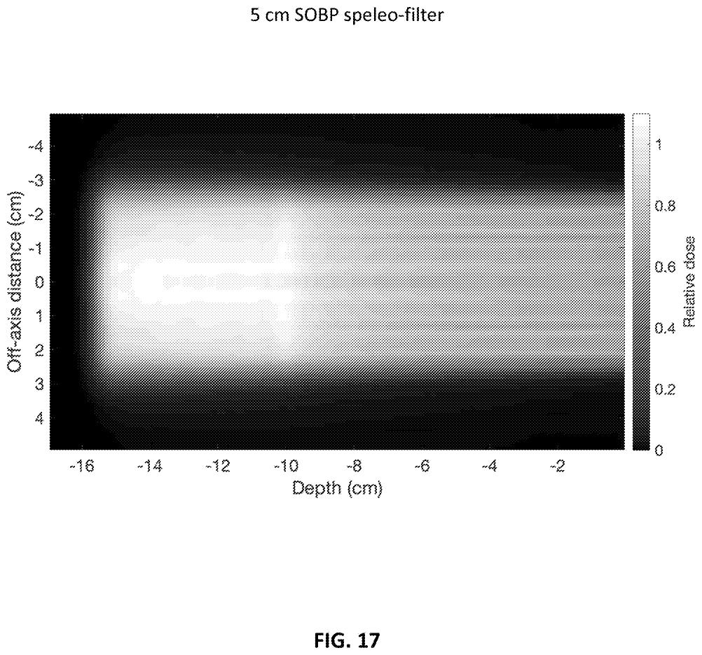

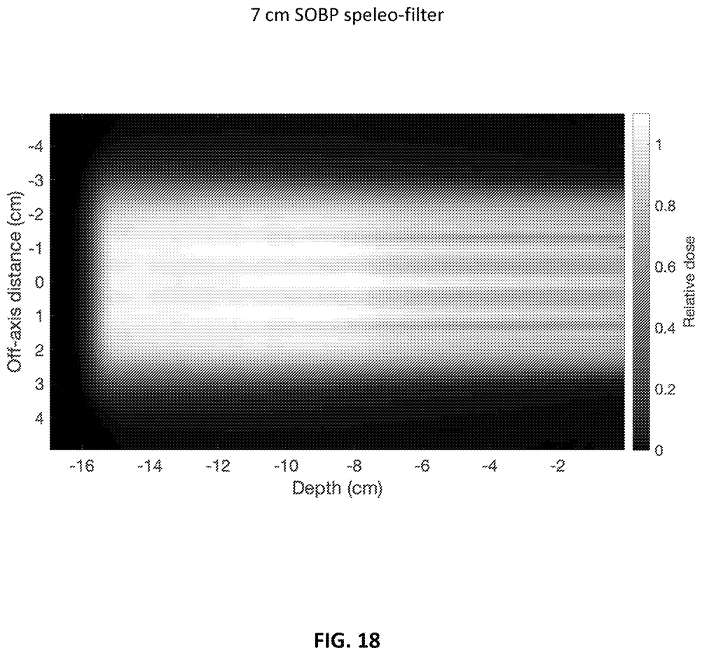

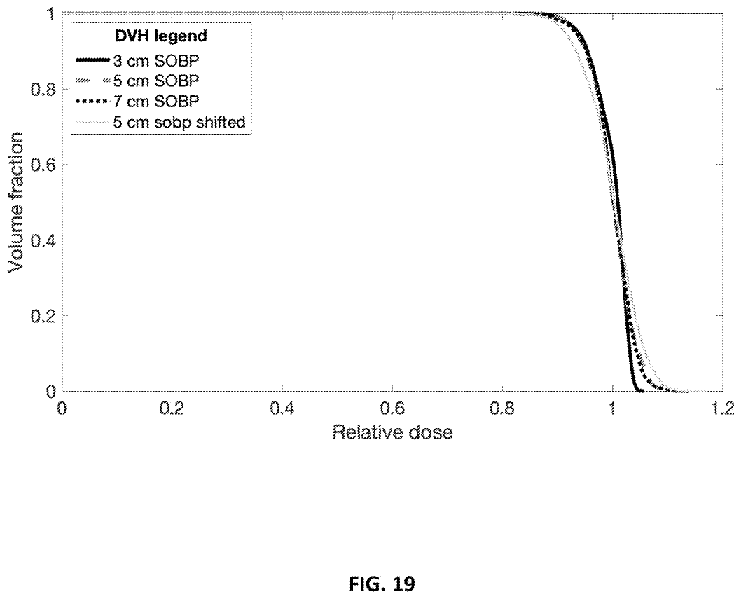

FIGS. 16-19 show the spatial distribution of a simulated dose profile. The relative dose is plotted so that the lightest portion correlates to the heaviest dose and the darkest portion correlates with the lightest dose. FIG. 16 shows the simulated distribution for a 3 cm speleo-filter. FIG. 17 shows the simulated distribution for a 5 cm speleo-filter, and FIG. 18 shows the simulated distribution for a 7 cm speleo-filter. FIG. 19 shows the volume fraction for these three cases in addition to a special case of off-setting the 5 cm speleo-filter upstream into the proton beam by 10 cm. The offset filter is shown as the light gray line in FIG. 19. There is little difference to be observed in this simulation amongst the filters, which indicates that the simulated proton beams which have passed through the filters have indeed been modified to achieve the desired dose.

FIG. 20 illustrates two example versions of speleo-filters used for experimental validation and treatment delivery both with and without a protective border. The speleo-filters can be a filter 100 as described herein. The unshielded filter 2010 example embodiments of filter 100 used a base 5 cm long by 5 cm wide and the columns were fabricated using additive 3D printing. The goal was a 5 cm SOBP dose. Alternatively, subtractive methods could also be employed to fabricate a speleo-filter. In the shielded filter 2020 embodiments of filter 100, the protected borders can be sidewalls 2030-1 to 2030-4, which can be fabricated to extend from the base so as to protect the columns from any damages that might result from any force or object striking from the lateral direction. Each sidewall can be of any dimension, and the height of the sidewalls 2030-1 to 2030-4 can be of the same or slightly greater height than the highest column.

FIGS. 21-23 show the measured and simulated dose profiles used to validate the Monte Carlo model for the SpeleoSolid, SpeleoPillar, and the SpeleoOptimized speleo-filters, as discussed above.

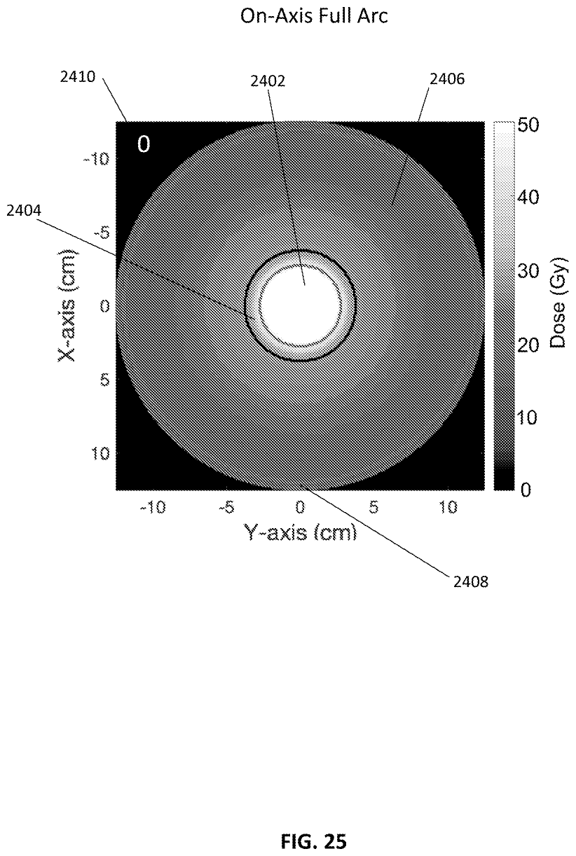

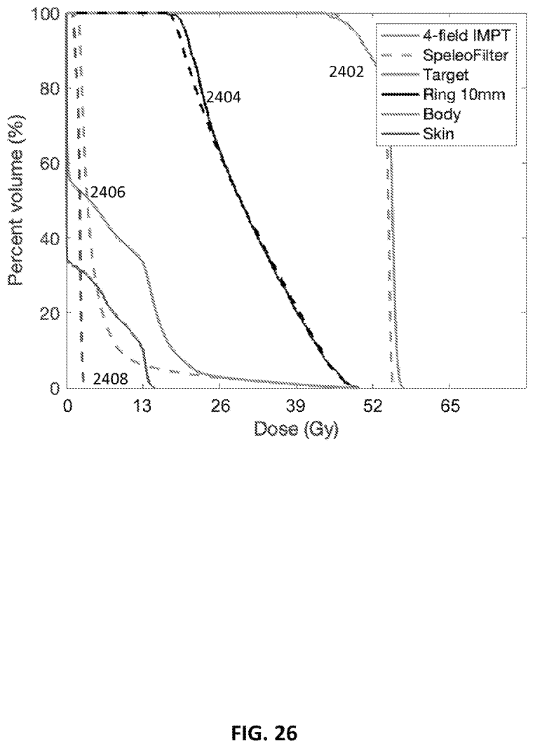

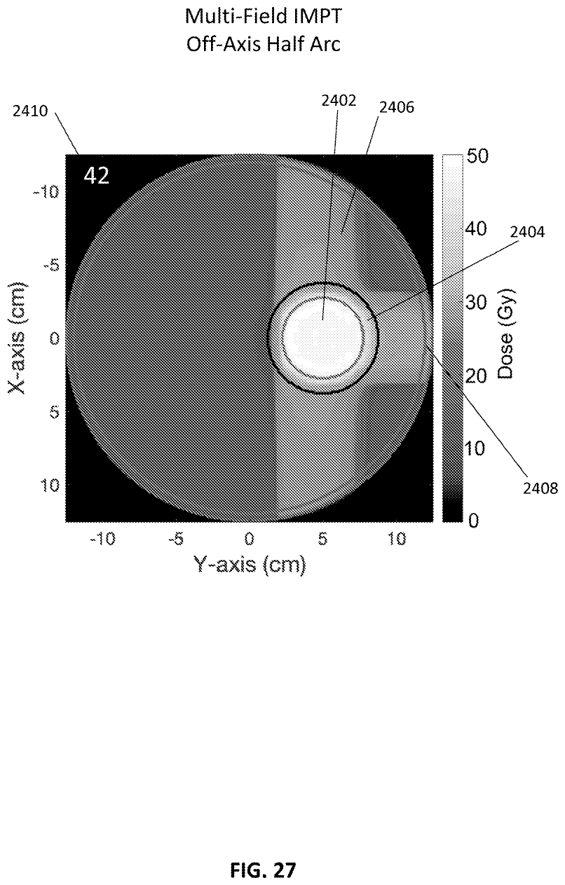

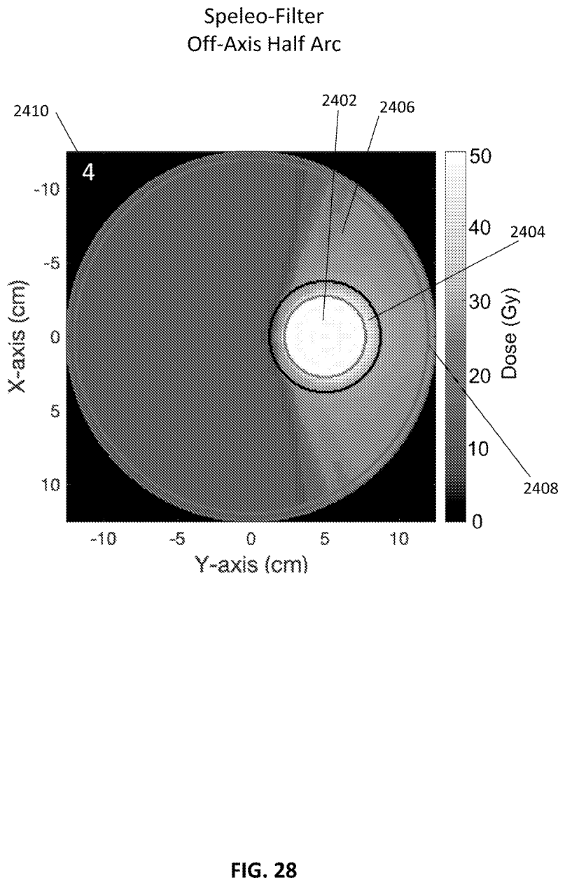

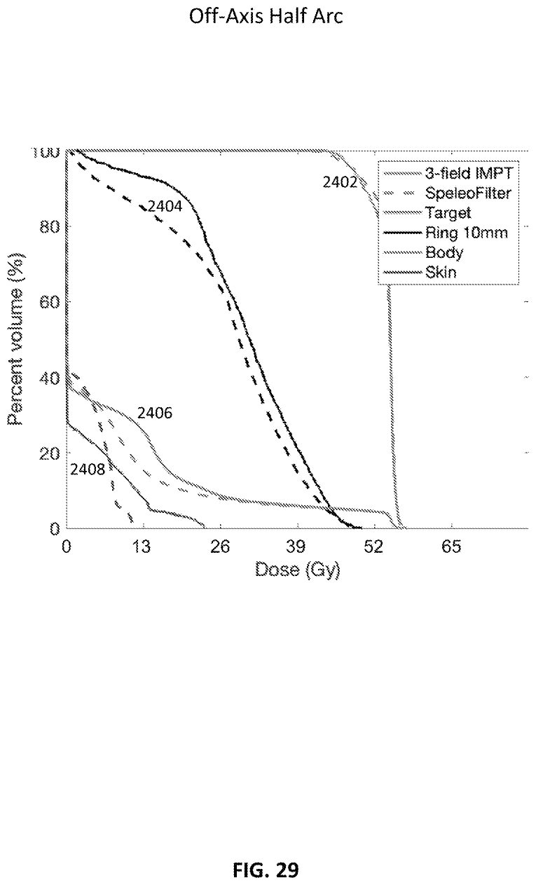

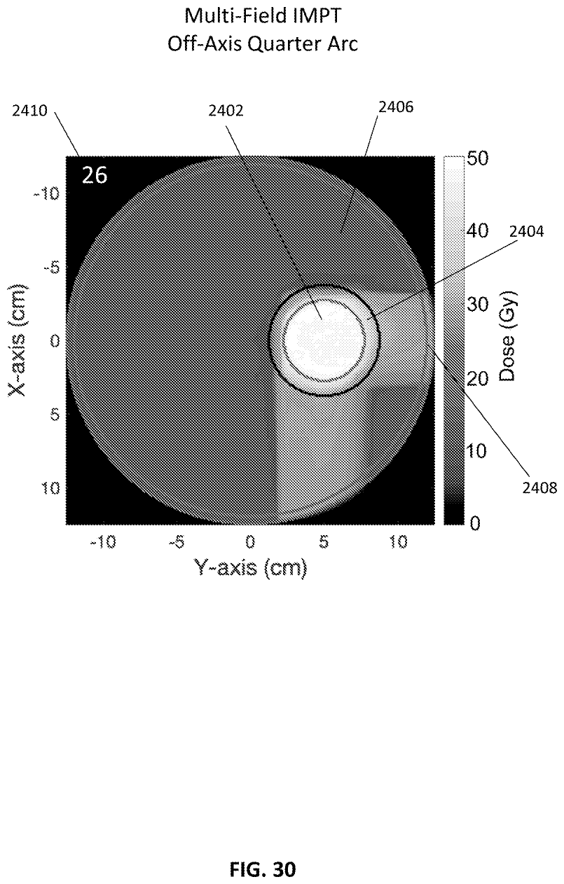

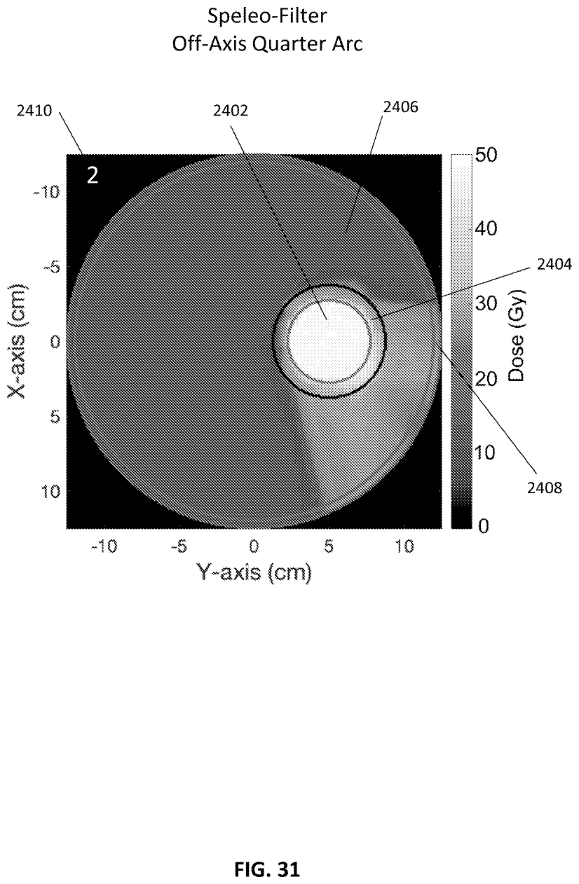

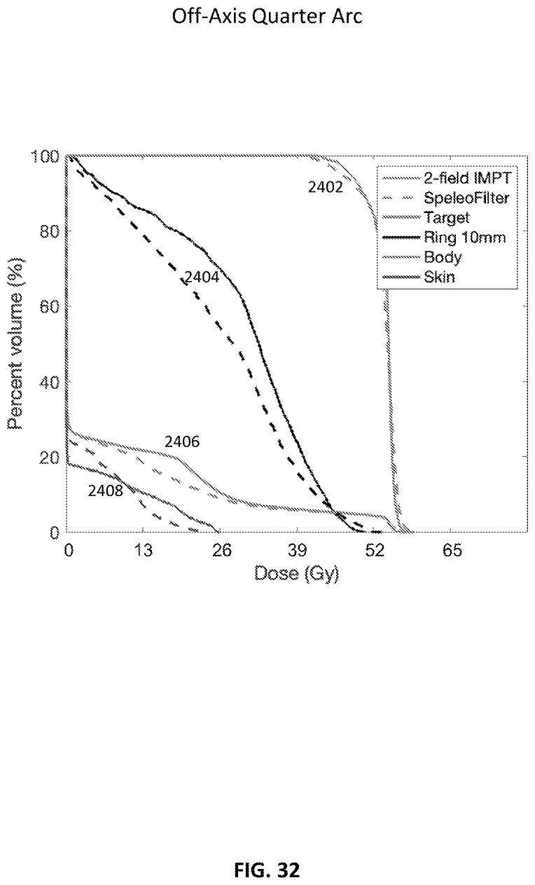

FIGS. 24-32 illustrate a comparison of the simulated dose between using multi-field IMPT and a speleo-filter from various arc treatments. In these figures, the heaviest dose (50 Gy) is the lightest color. The darker shades of grey represent the lower doses, down the lowest dose (0 Gy). For all these figures, the central portion (lightest color) of the simulated image is the target region 2402—the area inside the central circle. The near-target tissue 2404 lies in the 10 mm wide ring between the circle which bounds the target and the next largest circle. In these simulations, the near-target tissues 2404 is typically the second most exposed region after the target 2404. In addition, modelling for the skin 2408, between the outermost two circles in the images, and for the entire body 2406 was also performed. Also illustrated in FIGS. 24, 25, 27, 28, 30, and 31 is a charge number 2410, which appears in the upper lefthand corner of the image of these figures, and corresponds to the number of energy changes used. For the on-axis, full arc case intensity modulated proton therapy (IMPT) was simulated at four entry angles (at 0°, 90°, 180°, and 270°). The same on-axis, full arc case for the speleo-filter is shown in FIG. 25. Both of these full-arc examples show that the target receives the desired dose (approximately 50 Gy, in these examples) and that that dose is relatively uniform in the target region 2402. There is some residual absorption in the near-target region 2404 and also some in the skin 2408. FIG. 26 shows the dose volume histogram for the full arc cases with the speleo-filter results in the dashed line and the four-field IMPT in the solid line. The biggest difference between the full arc speleo-filter and the four-field IMPT occurs for the body 2406 and skin 2408. The off-axis three-field IMPT (FIG. 27) and half-arc speleo-filter (FIG. 28) are also compared in FIG. 29. In this case, the speleo-filter also shows lowered dose in the body 2406 and some of the skin 2408. FIGS. 31-33 compare the two-field IMPT case (FIG. 30) with the quarter arc speleo-filter case (FIG. 31). The resulting dose volume histogram for two-field IMPT and the quarter arc speleo-filter are shown in FIG. 32.

FIG. 33 illustrates a filter carousel, with, for example, three wheels, each wheel containing, in this example, four filters and one open slot. The filter carousel 3300 can have one filter wheel 3302, or several filter wheels 3302, 3304. The filter wheels 3302, 3304 can be held together by a frame 3310 or other supporting structure. The individual supporting structures for each filter can also not be a wheel, but any securing template or a filter itself. The frame 3310 can facilitate the rapid movement of a filter wheel 3302 out of or into the proton beam 110. The filter wheel 3302 can comprise multiple filters 3306, 3308 (five in the example illustrated) which can be rapidly and easily rotated into the proton beam 110 for filtering the beam.

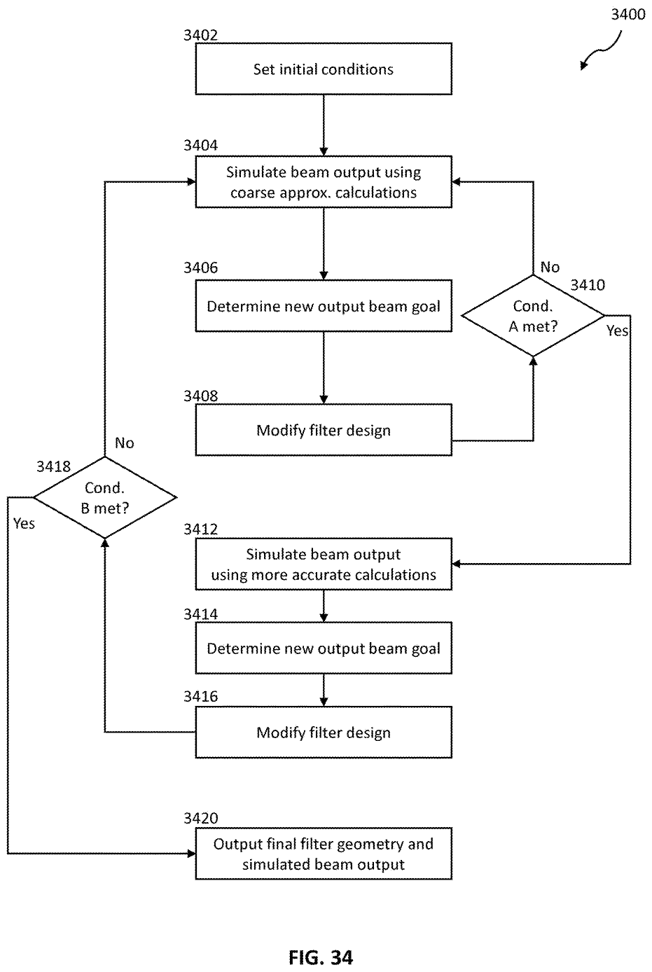

FIG. 34 illustrates an exemplary method 3400 for designing a speleo-filter (e.g., speleo-filter 100) for a specific designed absorbed dose, as a variation to the exemplary method described in FIG. 6. The initial conditions can be established at step 3402. As in step 610, the initial conditions comprise an initial material and geometry of the filter. The filter can be fabricated of a certain material which has certain parameters which describe the material's interaction with an ion beam, such as mass density, strength of nuclear scattering, and other parameters. The filter can comprise a base and a set of columns or pillars which extend from the base. The base and the columns can have dimensions: lengths, widths, and heights. Especially the heights of the columns can be varied in a set of calculations to estimate an output beam of protons, given an input beam. The initial conditions can comprise a height, length, width, and a location on the base for each column attached to the base, and for each column, the height can be varied as part of the process for optimizing the filter. The other filter dimensions can also be varied, but the examples described here vary only the height of each column, while keeping the locations, lengths, and widths fixed. In principal, these other dimensions can be varied in an analogous manner described for the column heights; however, the variation of the heights is more important than varying the other dimensions to properly optimize the output beam. The initial conditions can also comprise information about the input beam which interacts with the filter. The initial conditions can also comprise information about the desired output beam, for instance, the desired energy distribution of the beam and the desired spatial characteristics of the output beam, after passing through the filter. The initial conditions can further comprise information about the desired target.

The method 600 can simulate the output beam using a coarse approximation calculation at step 3404. This coarse approximation might not include all aspect of the beam interaction but can enable more rapid calculations. After this calculation has been completed, the output beam can be compared to the desired output beam, and a new output beam goal can be determined at step 3406. The filter design can be modified based on the new output beam goal at step 3408. The process can then evaluate whether a first condition or a first set of conditions have been met at step 3410, denoted in FIG. 34 as “Cond[ition] A met?”. This first set of conditions can comprise whether the coarse approximation calculations have been iterated more than a first number of times. This first set of conditions can also comprise whether the calculated beam output and the desired beam output match to within a first threshold. Other conditions can also be evaluated. If the first set of conditions has been met, then the method can proceed to step 3412, where the output beam can be simulated by more accurate simulations, for example, Monte Carlo methods. These calculations take more computer resources, and thus more time, but also can produce a more accurate prediction of the output beam. The example cited uses Monte Carlo methods, but other computational methods can also be used. The desired output beam can then be updated at step 3414 to the new output beam goal, and the filter design modified at step 3416 to more nearly achieve the output beam goal. Then at step 3418, a second set of conditions can be evaluated, denoted in the figure as “Cond[ition] B met?”. If the process has met this second set of conditions, then the last modified filter geometry can be sent to the user along with the simulated beam output and the desired beam output, for comparison at step 3420. If the second set of conditions are not met, then the method can continue at step 3404, with the last updated filter design and the new output beam goal. The second set of conditions can comprise whether the Monte Carlo simulation (or other more accurate, but more resource intensive simulation) has been iterated more than a second number times. The second set of conditions can also comprise whether the calculated beam output and the desired beam output match to within a second threshold. This second threshold can be the same as the first threshold for the internal loop, or it can be different than the threshold for the internal loop.

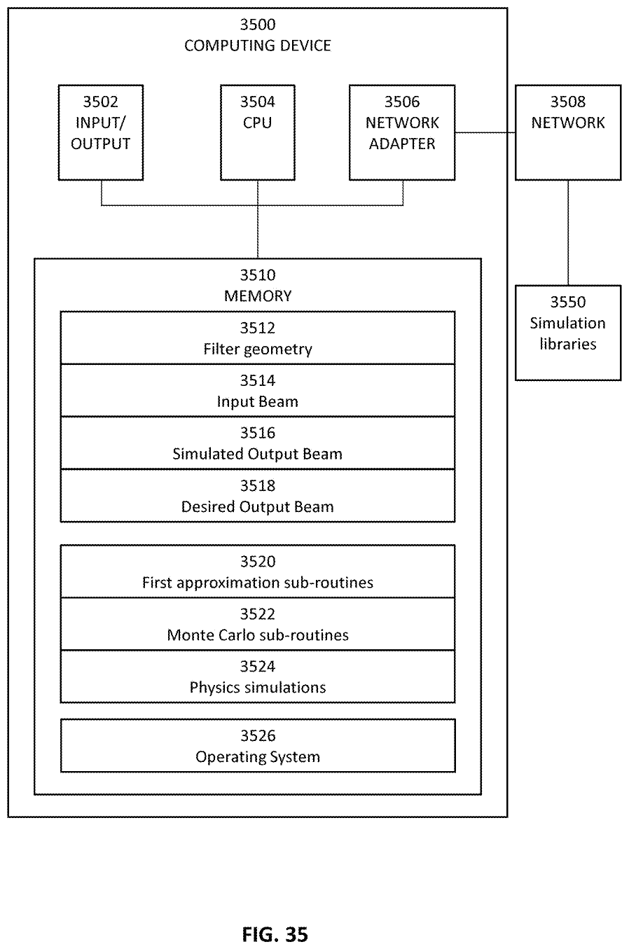

FIG. 35 illustrates a computer used for performing the calculations described elsewhere in this disclosure. A computing device 3500 can comprise one or more CPUs 3504, an input/output component 3502, a network adapter 3506, and memory 3510. The network adapter 3506 can connect to a network 3508, which can connect to many outside sources of information, for example simulation libraries 3550 for simulating the interaction of a proton beam with certain materials, or other resources, for instance other computer which can be configured to simulate interactions between protons and materials. Inside the memory 3510 of the computing device 3500 there can be sub-routines and simulations, for example sub-routines for calculating a coarse approximation of the interaction of a proton beam with a speleo-filter 3520, or sub-routines for performing Monte Carlo simulations 3522, or sub-routines for performing other physical simulations 3524. In addition, stored in memory, as part of these calculations there can reside filter geometry information 3512, input beam information 3514, output beam information 3516, and desired output beam information 3518. Additionally, the memory 3510 will also store an operating system 3526 for interacting with the various components of the computing device 3500.

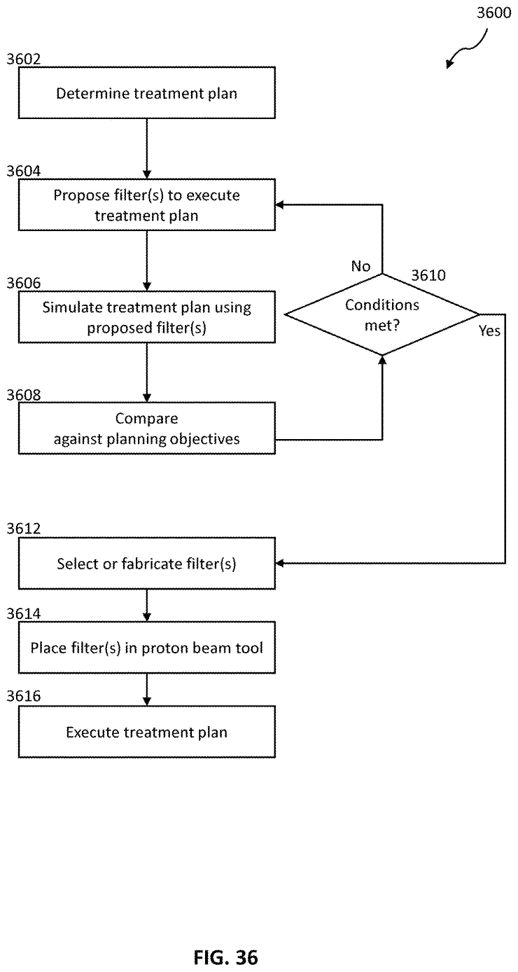

FIG. 36 illustrates an exemplary method of using the speleo-filter (or filters) in the treatment of a patient. The treatment method 3600 starts with determining the treatment plan 3602. The treatment plan comprises the appropriate dose of radiation, dosimetric goals to the target and healthy tissues, and where that radiation should be placed in the patient. For instance, in FIG. 9, the target 904 may be a tumor lodged inside a patient 902. Doctors may determine that a certain dose of radiation (e.g. ions or protons), which can be referred to as a specified, desired, intended, or planned dose distribution, can be used to destroy the tumor 904. The treatment plan will specify the amount of radiation that can be used to destroy that tumor 904, but without unduly irradiating other parts of the body of the patient 902. Once the required dose of radiation and the location are determined, then the planning phase shifts to how to apply that dose of radiation to the target 904. This part of the plan can include modeling how a proton beam can interact with a filter or a series of filters and can further involve irradiating the patent 902 from multiple locations at multiple angles, and also using multiple doses or energies of the proton beam. FIG. 9 illustrates an exemplary treatment plan showing irradiating the patient 902 from three distinct angles and at three distinct locations 906, 908, and 910 to maximize the dose received by the tumor 904, but to minimize the dose absorbed by the rest of the patient 902. This aspect of the treatment plan is determined at step 3604, by the interaction of the proton beam with the speleo-filter or filters. As noted elsewhere in this disclosure, a carousel wheel 3300 or other apparatus can be employed to quickly and easily shift filters 3306, 3308 into or out of the proton beam 110 to apply the correct dose to the patient 902. At step 3604, a set of filter or filters is proposed to implement the desired treatment plan. At step 3606, the chosen filters are used as part of a simulation of the treatment plan, given the beam characteristics and the filter characteristics. The simulated plan is then compared against the dosimetric planning goals at step 3608. How well the simulated treatment plan matches the desired planning goals is evaluated at step 3610. If the plan does not satisfy the dosimetric goals, then new filters can be proposed, at step 3604. Alternatively, a patient-specific filter can be determined during the planning step 3604. The process can be iterated until some set of conditions are met, such as, for example, a certain number of iterations has been exceeded, or a similarity between the desired treatment plan and the planning goals match to within a threshold value. Once the conditions have been met, the method can proceed to step 3612, where the filters can be selected from a known set of filters or the filters can be fabricated by, for instance, 3D printing of speleo-filters specific for this treatment plan. Alternatively, a set of pre-fabricated filters can also be employed. Each filter can be placed in the proton beam at step 3614. At step 3616, the patient can be irradiated by the proton beam through the filter. The execution of the treatment plan 3616 can comprise a single irradiation by a single proton beam through a single filter, but the execution of the treatment plan can also comprise multiple filters, multiple beams, and multiple irradiations from multiple directions. Thus a single filter can be used or multiple filters can be used. If multiple filters are used, each filter can be used with one single beam irradiation, followed by a change of filter to the next filter, and a new beam irradiation.

In example embodiments in accordance with the present invention, an ion beam filter for rotational arc therapy and ion beam delivery methods can comprise a base (e.g., base 102) and a plurality of pillars (e.g., pillars 104-1 to 104-N) extending from the base, wherein each pillar of the plurality of pillars has a height and dimensions perpendicular, or substantially perpendicular, or nonperpendicular, to the height.

In example embodiments in accordance with the present invention, a method of designing a filter with a base and a plurality of pillars extending from the base, each pillar having a height, the method can comprise setting initial conditions for an input ion beam for one or more ion beam angles or rotational arcs, a desired output ion beam distribution among one or more beam angles or rotational arcs, and dimensions for a filter comprising a plurality of pillars extending from a base. The method can also comprise modifying at least the height of each pillar of the plurality of pillars. The method can also comprise modeling the input ion beam passing through the filter apparatus and the ion beam's angle of incident on the patient among one or more beam angles or rotational arcs to determine an output ion beam. The method can also comprise comparing the determined output ion beam with the desired output ion beam or clinical objectives. The method can also comprise iterating the modifying, modeling, and comparing steps until the determined output ion beam matches the desired output ion beam to within a threshold or until a number of iterations has been exceeded. And the method can comprise creating and storing a file comprising the heights and locations of each pillar of the plurality of pillars. This method can further comprise fabricating a filter using the stored file.

In accordance with example embodiments of the present invention, an apparatus can comprise an ion beam therapy treatment apparatus for producing and aligning an ion beam among one or more beam angles or rotational arcs, and a filter (e.g., filter 100), wherein the filter resides in the ion beam path before the ion beam enters a patient.

In accordance with example embodiments of the present invention, a method of treating a patient can comprise determining an optimal radiation treatment plan for the patient, designing a filter whose physical characteristics enable a minimum number of energy changes required to carry out the optimal treatment plan, and executing the optimal treatment plan using the designed filter.

Example embodiments of an apparatus in accordance with the subject invention of this application can comprise a filter (e.g., filter 100) having a base and a plurality of pillars, or columns (e.g., pillars 104-1 to 104-N), extending from the base (e.g., base 102), wherein the one or more of the plurality of pillars are of varying height, enabling the filter to convert the energy spectrum associated with radiation as the radiation passes through the one or more of the plurality of pillars (see, e.g., FIG. 3). The filter is configured to modify the energy distribution of the radiation passing through the filter, resulting in a delivery of a dose distribution within a subject.

The plurality of pillars extending from the base can be aligned with the direction of the radiation while the radiation is delivered. The pillars can be guarded, or unguarded (see, e.g., FIG. 20). The radiation can comprise one or more beams of radiation. The filter can comprise a homogeneous material, wherein the filter is constructed from either a flat block or divergent block, wherein either the flat block or the divergent block comprises a homogenous block of material with implanted heterogeneities.

The filter can comprise one or more of the following materials: Vero, VeroClear, VeroUltraClear, VeroUltraClearS, VeroUltra, polylactic acid, Somos 9120, Somos WaterShed, SomosR WaterClear Ultra, Nylon 12 Carbon Fiber, Carbon Fiber, ABS-M30i, ULTEM resin or other polytherimide thermoplastics, photopolymers, acrylic, Carbon fiber, brass, nickel, aluminum, SolidWater, VirtualWater, graphite, and paraffin wax. The filter can be manually machined, or it can be robotically machined. The filter can be fabricated using a 3D printing technique, wherein the 3D printing technique comprises one or more of PolyJet, Selective Laser Sintering, Stereolithography, Fused Deposition Modeling, and Carbon digital light synthesis.

The dose distribution can comprise a desired (or specified or planned) dose distribution that is based on a geometry of the subject and the delivery of the radiation. The dose distribution can comprise a planned spatial distribution. The radiation passing through the filter results in a penetration depth profile shift in which multiple depths of a target associated with the subject can be reached by the radiation. The filter can be configured to produce a planned spatial distribution unique to a geometry of the subject and unique to the delivery of the radiation. The thickness of the material throughout the filter results in a spatially uniform particle energy spectrum.

The example apparatus can comprise a collimator connected to the filter, wherein the collimator comprises an energy-specific collimator. The collimator can be connected to the filter via a mounting apparatus. The mounting apparatus secures the filter such that the filter can be positioned before the collimating components of the collimator, or the filter can be positioned after the collimating components of the collimator. The filter, when connected to the collimator, can be movable, and the motion of the filter comprises one or more of a translation, a rotation, and an oscillation. The example apparatus can further comprise a carriage that holds the filter, and at least one more of the filter, and can position the filters in a path of the radiation. The position the filters at a desired location can be modified by adjusting the position of the filters such that the incidence of the radiation through the filter to the subject. The position of the filter can be changed as the dose distribution is delivered to the subject, and one or more filters can be switched to actively modify the radiation or be removed from the path of the radiation so as to no longer interact with the radiation.

The filter can be one of a library of filters that can be rapidly changed throughout a treatment cycle. The filter can be one of a plurality of filters, wherein the filters are organized in sets of nominal thicknesses and the targeted depth dose shape they produce. The filters can modify the radiation, resulting in the production of a desired polyenergetic spectrum.

An exemplary method in accordance with the present invention can be a method for optimizing example embodiments of a filter (e.g., filter 100). The method can comprise specifying one or more filters that results in a desired dose of radiation (which can be one or more beams of radiation) into a subject, wherein the radiation is incident from one or more directions; and wherein the specifying the one or more filters is based upon one or more optimization goals. The one or more optimization goals can comprise: the desired dose distribution, wherein the desired dose distribution is created by a spatially distributed energy distribution that achieves the desired dose distribution in the subject; dosimetric criteria comprising one or more of a target coverage, dose homogeneity, dose conformity and an organ dose limit; a constructability (e.g., is it possible to manufacture a filter for this particular purpose) of the one or more filters; a radiation definition comprising a number of arcs, an arc length, isocenters, spot configuration, and radiation output; and a filter position and orientation throughout a radiation delivery.

The method can further comprise iteratively optimizing the thickness distribution of the one or more filters on a base associated with each of the one or more filters, while accounting for the one or more filter's impact on the radiation from one or more directions. Each of the one or more filters can comprise a plurality of pillars (e.g., pillars 104-1 to 104-N) extending from the base (e.g., base 102), wherein the one or more of the plurality of pillars are of varying height, enabling the one or more filters to convert the energy spectrum associated with the radiation as it passes through the one or more of the plurality of pillars. The pillars can be shielded or unshielded (see, e.g., FIG. 20). The one or more filters can be configured to modify the energy distribution of radiation passing through the one or more filters, resulting in a delivery of a desired dose distribution within the subject. The one or more filters can be optimized based on the removal of material from the one or more filters.

The method of claim 26, wherein the method is based upon and accounts for a radiation delivery approach comprising one or more rotational arcs that relate to delivering a specified radiation dose into the subject, wherein setting an orientation of the one or more filters to one or more radiation directions augments the resulting energy distribution passing through the one or more filters for one or more radiation energies. The one or more filters can comprise a unique, patient-specific filter and the one or more radiation energies can be assigned to the one or more rotational arcs. The one or more filters comprise a general-purpose, non-patient-specific filters and one or more radiation energies are used to achieve the desired dose distribution into the subject.

The method can further comprise: using simultaneous gantry rotation to deliver the desired dose to the subject; applying a collimator to improve the delivery of the desired dose distribution in the subject for any radiation passing through the one or more filters; utilizing one or more radiation angles to deliver the desired dose distribution into the subject; deterministically modeling the transport of the radiation through the one or more filters to optimize a desired dose distribution to be delivered into the subject; stochastically modeling the transport of the radiation through the one or more filters to optimize a desired dose distribution to be delivered into the subject.

In other techniques related to the exemplary method, one of the one or more filters is a first filter, and can be superimposed on top of a second filter of the one or more filters. The method can also comprise rotating from one filter of the one or more filters to another of the one or more filters, using a carousel that retains the one or more filters.

In accordance with exemplary embodiments of the present invention, there is a method for delivering treatment radiation for a target volume in a subject using one or more filters (e.g., filter 100) that impact radiation, wherein the radiation can comprise one or more radiation beams. The method can comprise comprising defining a trajectory of the radiation through one or more filters into the subject, while moving a radiation source, one or more filters, and the subject to impart a planned dose distribution into the subject. The one or more filters comprise a base (e.g., base 102) and a plurality of pillars extending from the base (e.g., pillars 104-1 to 104-N), wherein the one or more of the plurality of pillars are of varying height. Defining the trajectory can further comprise defining the trajectory of the radiation through the one or more filters and a collimator into the subject, wherein the collimator comprises a device that interacts with a primary beam of the one or more radiation beams to attenuate the radiation to prevent it from reaching the subject. The trajectory can be based upon a span of locations wherein the start of the treatment radiation is directed from the radiation source toward the subject from a first position, and a subsequent instance of treatment radiation is directed from the radiation source towards the subject from a second position, wherein the trajectories are either coincident, or span one or more angular segments around the subject. The method can further comprise using a set of control points comprising a discrete set of angular segments or nominal gantry angles wherein the position and motion of a particular filter is defined.

The method can further comprise using the radiation source to delivers radiation through the filter resulting in a planned dose distribution into the subject, wherein the delivery originates from one or more positions that are variable, and the trajectory of the radiation into the subject is based upon the one or more positions of the radiation source and the subject. The movement of the filter with respect to the subject and the radiation source can be operable to remain fixed or dynamic relative to one or more radiation directions.

The method can further comprise: using one or more filters in which there is variation in one or more of an optimization of the intensities of the radiation; using of one or more filters for one or more directions; modifying the shape and energy of the incident radiation over at least a portion of its trajectory towards the subject; using radiation directed at one or more angles; using radiation of one or more energy; using one or more orientations of the filter, the one or more orientations corresponding to a direction of the radiation into the patient; using a modulation wheel, wherein a modulation wheel is known to those of ordinary skill, to create a desired depth dose profile in the subject; and using a ridge filter to create a desired depth dose profile in the subject.

We claim:

1. An apparatus comprising:
a filter having a base; and
a plurality of pillars extending from the base, wherein:
the one or more of the plurality of pillars are of varying height, enabling the filter to convert the energy spectrum associated with radiation as the radiation passes through the one or more of the plurality of pillars, and
the filter is configured to modify the energy distribution of the radiation
passing through the filter, resulting in a delivery of a dose distribution within a subject.

2. The apparatus of claim 1, wherein the plurality of pillars extending from the base are aligned with the direction of the radiation while the radiation is delivered.

3. The apparatus of claim 1, wherein the radiation comprises one or more beams of radiation.

4. The apparatus of claim 1, wherein the filter comprises a homogeneous material.

5. The apparatus of claim 1, wherein:
the filter is constructed from either a flat block or divergent block, wherein:
the flat block comprises a homogenous material with implanted heterogeneities, and
the divergent block comprises a homogenous material with implanted heterogeneities.

6. The apparatus of claim 1, wherein the filter comprises one or more of the following materials: Vero, VeroClear, VeroUltraClear, VeroUltraClearS, and VeroUltra.

7. The apparatus of claim 1, wherein the filter comprises one or more of the following materials: polylactic acid, Somos 9120, Somos Watershed, SomosR WaterClear Ultra, Nylon 12 Carbon Fiber, Carbon Fiber, ABS-M30i, ULTEM resin or other polytherimide thermoplastics, photopolymers, acrylic, Carbon fiber, brass, nickel, aluminum, SolidWater, VirtualWater, graphite, and paraffin wax.

8. The apparatus of claim 1, wherein the filter is fabricated using a 3D printing technique.

9. The apparatus of claim 8, wherein the 3D printing technique comprises one or more of Poly Jet, Selective Laser Sintering, Stereolithography, Fused Deposition Modeling, and Carbon digital light synthesis.

10. The apparatus of claim 1, wherein the dose distribution comprises a desired dose distribution that is based on a geometry of the subject and the delivery of the radiation.

11. The apparatus of claim 1, wherein the dose distribution comprises a planned spatial distribution.

12. The apparatus of claim 1, wherein the radiation passing through the filter results in a penetration depth profile shift in which multiple depths of a target associated with the subject can be reached by the radiation.

13. The apparatus of claim 1, wherein the filter is configured to produce a planned spatial distribution unique to a geometry of the subject and unique to the delivery of the radiation.

14. The apparatus of claim 1, wherein the thickness of the material throughout the filter results in a spatially uniform particle energy spectrum.

15. The apparatus of claim 1, wherein the apparatus is manually machined.

16. The apparatus of claim 1, wherein the apparatus is robotically machined.

17. The apparatus of claim 1, 1 further comprises a collimator connected to the filter.

18. The apparatus of claim 16, wherein the collimator comprises an energy-specific collimator.

19. The apparatus of claim 16, wherein the collimator is connected to the filter via a mounting apparatus.

20. The apparatus of claim 19, wherein the mounting apparatus 16secures the filter such that it is positioned before the collimating components of the collimator.

21. The apparatus of claim 19, wherein the mounting apparatus 16secures the filter such that it is positioned after the collimating components of the collimator.

22. The apparatus of claim 19, wherein:
the filter, when connected to the collimator, is movable, and
the motion of the filter comprises one or more of a translation, a rotation, and an oscillation.

23. The apparatus of claim 1, wherein the apparatus further comprises a carriage that holds the filter, and at least one more of the filter, and positions the filters in a path of the radiation, wherein:
the position the filters at a desired location can be modified by adjusting the position of the filters such that the incidence of the radiation through the filter to the subject,
the position of the filters can be changed as the dose distribution is delivered to the subject, and
one or more filters can be switched to actively modify the radiation or be removed from the path of the radiation so as to no longer interact with the radiation.

24. The apparatus of claim 1, wherein the filter can be one of a library of filters that can be rapidly changed throughout a treatment cycle.

25. The apparatus of claim 1, wherein:
the filter is one of a plurality of filters,
the filters are organized in sets of nominal thicknesses and the targeted depth dose shape they produce, and
the filters modify the radiation, resulting in the production of a desired polyenergetic spectrum.

26. A method for optimizing a filter, comprising:
specifying one or more filters that results in a desired dose of radiation into a subject, wherein:
the radiation is incident from one or more directions,
the specifying the one or more filters is based upon one or more
optimization goals; and
iteratively optimizing the thickness distribution of the one or more filters on a base associated with each of the one or more filters, while accounting for
the one or more filter's impact on the radiation from one or more directions, wherein:
each of the one or more filters comprise a plurality of pillars extending from the base, wherein the one or more of the plurality of pillars are of varying height, enabling the one or more filters to convert the energy spectrum associated with the radiation as it passes through the one or more of the plurality of pillars, and
the one or more filters are configured to modify the energy distribution of radiation passing through the one or more filters, resulting in a delivery of a desired dose distribution within the subject.

27.-43. (canceled)

44. A method for delivering treatment radiation for a target volume in a subject using one or more filters that impact radiation, the method comprising:
defining a trajectory of the radiation through one or more filters into the subject, while moving a radiation source, one or more filters, and the subject to impart a planned dose distribution into the subject, wherein the one or more filters comprise a base and a plurality of pillars extending from the base, and the one or more of the plurality of pillars are of varying height; and
using the radiation source to deliver radiation through the filter resulting in a planned dose distribution into the subject, wherein:
the delivery originates from one or more positions that are variable, and
the trajectory of the radiation into the subject is based upon the one or more positions of the radiation source and the subject.

45.-57. (canceled)
